(19) United States
(12) Patent Application Publication
(10) Pub. No.: US 2024/0408387 A1
(43) Pub. Date: Dec. 12, 2024

(54) COMBINATION ELECTRICAL THERAPY WITH OTHER CANCER THERAPIES

(71) Applicant: Boston Scientific Scimed, Inc., Maple Grove, MN (US)

(72) Inventor: Brian L. Schmidt, White Bear Lake, MN (US)

(73) Assignee: MAYO FOUNDATION FOR MEDICAL EDUCATION AND RESEARCH, Rochester, MN (US)

(21) Appl. No.: 18/734,618

(22) Filed: Jun. 5, 2024

Related U.S. Application Data
(60) Provisional application No. 63/471,652, filed on Jun. 7, 2023.

(51) Int. Cl. A61N 1/36; A61K 35/12; A61K 45/06; A61N 1/05

(52) U.S. Cl. CPC A61N 1/36002; A61K 35/12; A61K 45/06; A61N 1/05

(57) ABSTRACT

Embodiments herein relate to methods, devices, and systems for the treatment of cancer tumor by combining TTF electrical field therapy with other types of cancer therapies. In an embodiment, a method for providing cancer therapy is included, the method applying one or more TTF electrical fields at or near a site of a cancerous tumor, a surgical excision site, and/or a targeted therapy site of a subject and administering a non-electrical field based cancer therapy to the subject, wherein applying the TTF electrical fields is performed before, after, and/or simultaneously with administering the non-electrical field based cancer therapy. Other embodiments are also included herein.

This application claims the benefit of U.S. Provisional Application No. 63/471,652, filed Jun. 7, 2023, the content of which is herein incorporated by reference in its entirety.

### FIELD

Embodiments herein relate to methods, devices, and systems for the treatment of cancer by combining TTF electrical field therapy with other types of cancer therapies.

### BACKGROUND

According to the American Cancer Society, cancer accounts for nearly 25% of the deaths that occur in the United States each year. The current standard of care for cancerous tumors can include first-line therapies such as surgery, radiation therapy, and chemotherapy.

Cancerous tumors can form if one normal cell in any part of the body mutates and then begins to grow and multiply too much and too quickly. Cancerous tumors can be a result of a genetic mutation to the cellular DNA or RNA that arises during cell division, an external stimulus such as ionizing or non-ionizing radiation, exposure to a carcinogen, or a result of a hereditary gene mutation. Regardless of the etiology, many cancerous tumors are the result of unchecked rapid cellular division.

All actively dividing somatic cells undergo cellular division through the cell cycle, including many types of cancerous cells. Actively dividing cells move through two main phases of the cell cycle: interphase and the M phase. During interphase, the longest phase of the cell cycle, an individual cell begins doubling in size and replicating its DNA in preparation for cellular division. Interphase can be broken down into three discrete phases in the following order: the gap phase 1, or G1 phase; the synthesis phase, or S phase; and the gap phase 2, or G2 phase. In the G1 phase, the all of the cellular contents except for the chromosomes are duplicated and the cell begins to double its size. During the S phase, DNA synthesis replicates the chromosomes to form two sister chromatids for each chromosome in the cell. During the G2 phase, the cell continues its growth and prepares the cell and chromosomes for the M phase.

During the M phase, the cell exits interphase and begins the process of mitosis, or nuclear division, which includes separation of the sister chromatids. The M phase ends with cytokinesis, or cytoplasmic division. Mitosis includes four basic phases: prophase, metaphase, anaphase, and telophase. During prophase, the chromosomes start to condense and the nuclear membrane surrounding the nucleus disappears. The mitotic spindle also begins to form during prophase. The mitotic spindle includes a self-organized bipolar array of microtubules and centrosomes. Microtubules are generally formed from the polymerization of the highly polar alpha-tubulin and beta-tubulin proteins. Centrosomes are similarly protein-based organelles, two of which migrate to opposite sides of the dividing cell at this phase. The negatively charged end of the microtubules attach to the centrosomes. The positively charged end of the microtubules radiate toward the equator of the dividing cell where they eventually attach to a kinetochore of each sister chromatid. Metaphase can be defined by all chromosomes being aligned at the equator of the dividing cell and bound in the mitotic spindle. An equal number of sister chromatids are then pulled toward opposite ends of the cell during anaphase. Once all chromosomes have been separated, the process of telophase begins, where the cell membrane begins to form a cleavage furrow between the two newly forming sister cells, and cell division becomes complete once the cells physically separate from one another in a process called cytokinesis.

### SUMMARY

Embodiments herein relate to methods, devices, and systems for the treatment of cancer by combining TTF electrical field therapy with other types of cancer therapies. In a first aspect, a method for providing cancer therapy can be included. The method can include applying one or more TTF electrical fields at or near a site of a cancerous tumor, a surgical excision site, and/or a targeted therapy site of a subject. The method can also include administering a non-electrical field based cancer therapy to the subject. Applying the TTF electrical fields can be performed before, after, and/or simultaneously with administering the non-electrical field based cancer therapy.

In a second aspect, in addition to one or more of the preceding or following aspects, or in the alternative to some aspects, applying the TTF electrical fields can be performed before administering the non-electrical field based cancer therapy.

In a third aspect, in addition to one or more of the preceding or following aspects, or in the alternative to some aspects, applying the TTF electrical fields can be performed both before and after administering the non-electrical field based cancer therapy.

In a fourth aspect, in addition to one or more of the preceding or following aspects, or in the alternative to some aspects, applying the TTF electrical fields can be performed after administering the non-electrical field based cancer therapy.

In a fifth aspect, in addition to one or more of the preceding or following aspects, or in the alternative to some aspects, applying the TTF electrical fields can be performed simultaneously with administering the non-electrical field based cancer therapy.

In a sixth aspect, in addition to one or more of the preceding or following aspects, or in the alternative to some aspects, the TTF electrical fields can be applied at a first intensity simultaneously with administering the non-electrical field based cancer therapy and then at a second intensity after administering the non-electrical field based cancer therapy ceases.

In a seventh aspect, in addition to one or more of the preceding or following aspects, or in the alternative to some aspects, the TTF electrical fields can be applied for a first time period simultaneously with administering the non-electrical field based cancer therapy and then for a second time period after administering the non-electrical field based cancer therapy ceases, wherein the second time period can be longer than the first time period.

In an eighth aspect, in addition to one or more of the preceding or following aspects, or in the alternative to some aspects, administering a non-electrical field based cancer therapy to the subject includes administering a chemotherapeutic agent.

In a ninth aspect, in addition to one or more of the preceding or following aspects, or in the alternative to some aspects, the chemotherapeutic agent can be at least one of an antimitotic agent, an alkylating agent, an antimetabolite, a kinase inhibitor, a proteasome inhibitor, a histone deacetylase inhibitor, a topoisomerase inhibitor, a poly (ADP-ribose) polymerase (PARP) inhibitor, an antitumor antibiotic, or a retinoid.

In a tenth aspect, in addition to one or more of the preceding or following aspects, or in the alternative to some aspects, administering a non-electrical field based cancer therapy to the subject includes administering an immunotherapeutic agent to the subject.

In an eleventh aspect, in addition to one or more of the preceding or following aspects, or in the alternative to some aspects, the immunotherapeutic agent can be at least one of a monoclonal antibody, an adoptive cell therapeutic agent, a cancer vaccine, an immunomodulatory agent, or a cytokine.

In a twelfth aspect, in addition to one or more of the preceding or following aspects, or in the alternative to some aspects, administering a non-electrical field based cancer therapy to the subject includes administering a hormone/biologic therapeutic agent to the subject.

In a thirteenth aspect, in addition to one or more of the preceding or following aspects, or in the alternative to some aspects, the hormone/biologic therapeutic agent can be at least one of a progestin, a steroidal antiestrogen, a luteinizing hormone-releasing hormone agonist, a gonadotropin-releasing hormone agonist, an antiandrogen, an aromatase inhibitor, a selective estrogen receptor modulator, a corticosteroid, a somatostatin analogue, a prolactin lowering agent, or a thyrotropin stimulating hormone agonist.

In a fourteenth aspect, in addition to one or more of the preceding or following aspects, or in the alternative to some aspects, administering a non-electrical field based cancer therapy to the subject includes administering a telomerase inhibition agent to the subject.

In a fifteenth aspect, in addition to one or more of the preceding or following aspects, or in the alternative to some aspects, the telomerase inhibition agent can be a telomerase inhibitor, an alternative lengthening of telomeres (ALT) inhibitor, or a telomere-targeting agent.

In a sixteenth aspect, in addition to one or more of the preceding or following aspects, or in the alternative to some aspects, administering a non-electrical field based cancer therapy to the subject includes administering radiation to the subject.

In a seventeenth aspect, in addition to one or more of the preceding or following aspects, or in the alternative to some aspects, the radiation can be administered at a radiation dose selected from a range of between 1 Gy to 80 Gy.

In an eighteenth aspect, in addition to one or more of the preceding or following aspects, or in the alternative to some aspects, administering a non-electrical field based cancer therapy to the subject includes applying subzero temperatures to the subject.

In a nineteenth aspect, in addition to one or more of the preceding or following aspects, or in the alternative to some aspects, the subzero temperatures can be at a temperature selected from a range of between −10° C. to −200° C.

In a twentieth aspect, in addition to one or more of the preceding or following aspects, or in the alternative to some aspects, administering a non-electrical field based cancer therapy to the subject includes transplanting one or more stem cells into the subject.

In a twenty-first aspect, in addition to one or more of the preceding or following aspects, or in the alternative to some aspects, administering a non-electrical field based cancer therapy to the subject includes performing surgery on the subject.

In a twenty-second aspect, in addition to one or more of the preceding or following aspects, or in the alternative to some aspects, the surgery can be cancerous tumor excision surgery.

In a twenty-third aspect, in addition to one or more of the preceding or following aspects, or in the alternative to some aspects, administering a non-electrical field based cancer therapy to the subject includes performing laser therapy.

In a twenty-fourth aspect, in addition to one or more of the preceding or following aspects, or in the alternative to some aspects, the laser therapy includes applying one or more laser beams to the subject at a wavelength of 600 nm to 1200 nm.

In a twenty-fifth aspect, in addition to one or more of the preceding or following aspects, or in the alternative to some aspects, administering a non-electrical field based cancer therapy to the subject includes administering at least one selected from the group consisting of chemotherapy, radiation therapy, cryotherapy, surgery, immunotherapy, photodynamic therapy, stem cell transplant therapy, hormone/biological therapy, telomerase inhibition therapy, and gene therapy.

In a twenty-sixth aspect, in addition to one or more of the preceding or following aspects, or in the alternative to some aspects, the one or more TTF electrical fields can be applied at frequencies selected from a range of 100 kHz to 300 kHz.

In a twenty-seventh aspect, in addition to one or more of the preceding or following aspects, or in the alternative to some aspects, the one or more TTF electrical fields can be generated using an external device.

In a twenty-eighth aspect, in addition to one or more of the preceding or following aspects, or in the alternative to some aspects, the one or more TTF electrical fields can be applied at field strengths of 1 V/cm to 10 V/cm.

In a twenty-ninth aspect, in addition to one or more of the preceding or following aspects, or in the alternative to some aspects, the one or more TTF electrical fields can be applied at field strengths of 3 V/cm to 5 V/cm.

In a thirtieth aspect, in addition to one or more of the preceding or following aspects, or in the alternative to some aspects, the one or more TTF electrical fields can be applied using electrodes that can be external to the patient.

In a thirty-first aspect, in addition to one or more of the preceding or following aspects, or in the alternative to some aspects, the one or more TTF electrical fields can be applied using electrodes that can be on or over skin of the patient.

In a thirty-second aspect, in addition to one or more of the preceding or following aspects, or in the alternative to some aspects, the one or more TTF electrical fields can be applied using electrodes that can be applied transcutaneously.

In a thirty-third aspect, in addition to one or more of the preceding or following aspects, or in the alternative to some aspects, the one or more TTF electrical fields can be applied using electrodes that can be implanted within the patient.

In a thirty-fourth aspect, in addition to one or more of the preceding or following aspects, or in the alternative to some aspects, the one or more TTF electrical fields can be generated using an implanted device.

In a thirty-fifth aspect, a method of treating a cancerous tumor can be included. The method can include applying one or more TTF electrical fields at or near the site of the cancerous tumor and administering a non-electrical field based cancer therapy to the subject. Applying the TTF electrical fields can be performed before, after, and/or simultaneously with administering the non-electrical field based cancer therapy.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following drawings, in which:

FIG. 1 is a schematic view of an exemplary cell cycle in accordance with various embodiments herein.

FIG. 2 is a schematic view of the M-phase in a healthy eukaryotic cell in accordance with various embodiments herein.

FIG. 3 is a schematic view of M-phase in a cancerous eukaryotic cell in accordance with various embodiments herein.

FIG. 4 is a schematic view of a cancer therapy method in accordance with various embodiments herein.

FIG. 5 is a schematic view of a cancer therapy method in accordance with various embodiments herein.

FIG. 6 is a schematic view of a cancer therapy method in accordance with various embodiments herein.

FIG. 7 is a schematic view of a cancer therapy method in accordance with various embodiments herein.

FIG. 8 is a schematic view of a cancer therapy method in accordance with various embodiments herein.

FIG. 9 is a schematic view of a cancer therapy method in accordance with various embodiments herein.

FIG. 10 is a schematic view of a cancer therapy method in accordance with various embodiments herein.

FIG. 11 is a schematic view of a cancer therapy method in accordance with various embodiments herein.

FIG. 12 is a schematic view of a cancer therapy method in accordance with various embodiments herein.

FIG. 13 is a schematic view of a cancer therapy method in accordance with various embodiments herein.

FIG. 14 is a schematic view of a cancer therapy method in accordance with various embodiments herein.

FIG. 15 is a schematic view of a cancer therapy method in accordance with various embodiments herein.

FIG. 16 is a schematic view of a cancer therapy method in accordance with various embodiments herein.

FIG. 17 is a schematic view a medical device in accordance with various embodiments herein.

FIG. 18 is a schematic view a medical device in accordance with various embodiments herein.

FIG. 19 is a schematic view a medical device in accordance with various embodiments herein.

FIG. 20 is a schematic view a medical device in accordance with various embodiments herein.

FIG. 21 is a schematic cross-sectional view of medical device in accordance with various embodiments herein.

FIG. 22 is a schematic diagram of components of a medical device in accordance with various embodiments herein.

While embodiments are susceptible to various modifications and alternative forms, specifics thereof have been shown by way of example and drawings and will be described in detail. It should be understood, however, that the scope herein is not limited to the particular embodiments described. On the contrary, the intention is to cover modifications, equivalents, and alternatives falling within the spirit and scope herein.

### DETAILED DESCRIPTION

As referenced above, many cancerous tumors can result from unchecked rapid cellular division. Some therapies to treat cancerous tumors can include surgery, radiation therapy, and chemotherapy. However, many therapies may not provide a desired level of efficacy. However, combining TTF (tumor treating fields) electrical field therapy with a non-electrical field therapy can offer enhanced treatment efficacy. As such, embodiments herein include combination therapy including, for example, the application of TTF electrical fields in combination with another cancer therapy.

The application of alternating TTF electrical fields (such as at specific field strengths described herein) can disrupt mitosis within a cancerous cell(s) and is believed to occur by interfering with the dipole alignment of key proteins involved in cellular division; tubulin and septin in particular. The polymerization of tubulin proteins that form microtubule spindle fibers can be disrupted, thus preventing the formation of spindle fibers required for chromosome separation. This can halt cellular division at the metaphase stage of mitosis. In some instances an alternating TTF electrical field can halt polymerization of already growing spindle fibers, leading to incomplete spindles and unequal chromosome separation during anaphase, should the cell survive that long. In each case, halting microtubule spindle formation and unequal chromosome separation during anaphase caused by incomplete polymerization of microtubules, can result in apoptosis (i.e., programmed cell death). It will further be appreciated that in some embodiments, alternating TTF electrical fields can disrupt mitosis by interfering with proteins involved in the formation of the contractile ring that is responsible for generating the constricting force when two daughter cells are separated. Various proteins involved in the formation of the contractile ring can include, but are not to be limited to F-actin, myosin-2, anillin, one or more septins, Rho, profilin, cofilin, and male germ cell Ras-related C3 botulinum toxin substrate GTPase activating proteins (MgcRacGAP).

Embodiments herein include combination therapy including, for example, the application of TTF electrical fields in combination with another cancer therapy. For example, in some embodiments, chemotherapeutic agents can be administered in combination with (before, after, and/or simultaneously) TTF electrical field therapy. In some embodiments, nanoparticles can be administered in combination with (before, after, and/or simultaneously) TTF electrical field therapy. In other embodiments, surgery can be performed before or after TTF electrical field therapy. In some embodiments, cryotherapy can be administered in combination with (before, after, and/or simultaneously) TTF electrical field therapy. In some embodiments, radiation therapy can be administered in combination with (before, after, and/or simultaneously) TTF electrical field therapy. In other embodiments, immunotherapeutic agents can be administered in combination with (before, after, and/or simultaneously) TTF electrical field therapy. In some embodiments, hormone modulators or biologic therapeutic agents can be administered in combination with (before, after, and/or simultaneously) TTF electrical field therapy. In other embodiments, photodynamic therapy can be administered in combination with (before, after, and/or simultaneously) TTF electrical field therapy. In some embodiments, gene therapy can be administered in combination with (before, after, and/or simultaneously) TTF electrical field therapy. In some embodiments, telomerase inhibition therapy, can be administered in combination with (before, after, and/or simultaneously) TTF electrical field therapy. In some embodiments, TTF electrical field therapy can also be combined with electrical field therapy that is not TTF electrical field therapy.

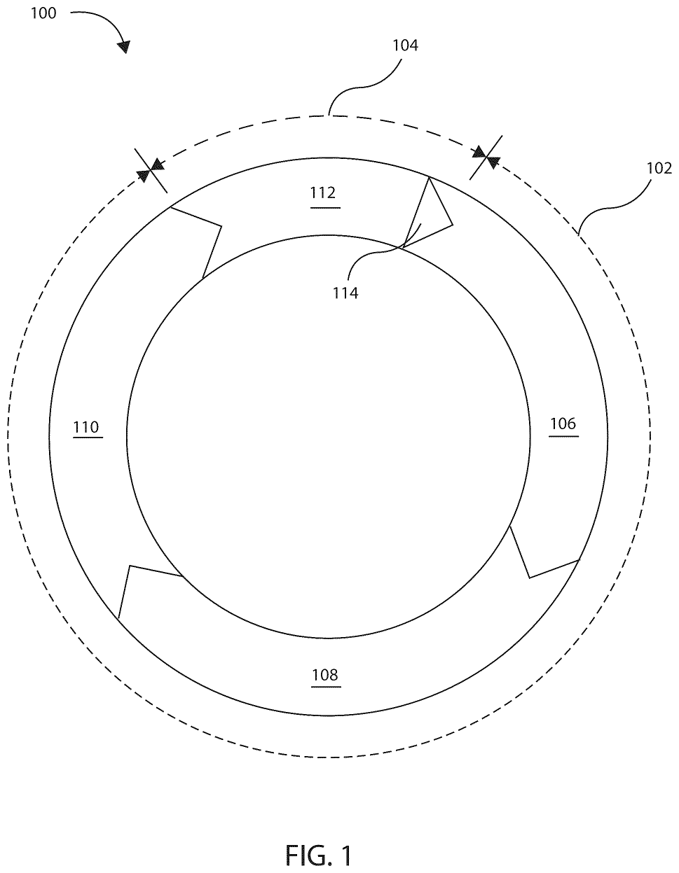

Referring now to FIG. 1, a schematic view of an exemplary eukaryotic cell cycle 100 is shown in accordance with the embodiments herein. A eukaryotic cell cycle can be broken into two major phases, including interphase 102 and the M phase 104. Interphase includes three key phases, including the G1 phase 106 (i.e., the gap phase 1); the S phase 108 (i.e., the synthesis phase); and the G2 phase 110 (i.e., the gap phase 2). During the G1 phase 106, the cell prepares itself for doubling by beginning the process of duplicating all of its cellular contents, exclusive of the chromosomes. During the S phase 108, the cell synthesizes new DNA through the process of DNA replication to form two sister chromatids for each chromosome in the cell. During the G2 phase 110, the cell continues its growth and it synthesizes the proteins required by the cell for the M phase.

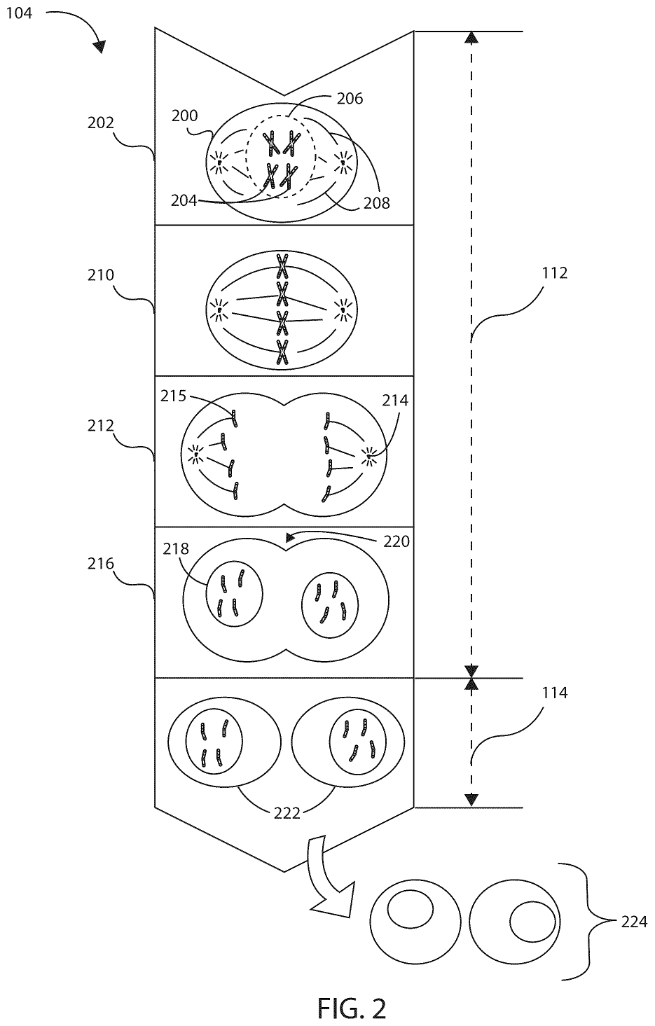

The M phase of the cell cycle 100 consists of two key phases: mitosis 112 and cytokinesis 114. Mitosis 112 is the process of nuclear division, and cytokinesis 114 is the process of cytoplasmic division. Referring now to FIG. 2, a schematic view of the M phase 104 in an exemplary eukaryotic cell cycle is shown in accordance with the embodiments herein. For healthy exemplary cell 200, mitosis begins at the process of prophase 202, where the chromosomes 204 begin to condense to form a pair of identical sister chromatids (sister chromatids 215 will be discussed below), the nuclear envelope 206 surrounding the nucleus disappears, and the spindle apparatus, including the spindle fibers 208, begin to form. Prophase 202 is followed by metaphase 210, where the chromosomes 204 line up on the equatorial plane of the dividing cell and become bound by the spindle fibers 208 at the kinetochores of each chromosome. The chromosomes 204 are then separated during anaphase 212 by the action of the spindle fibers 208 pulling each sister chromatid 215 towards each centrosome 214 and to opposite poles of the dividing cell. During telophase 216, a nuclear membrane 218 reforms around each set of chromosomes 204 at the opposite poles of the dividing cell, and a cleavage furrow 220 begins to form between the two halves of the dividing cell. The final step in the cell cycle is the step of cytokinesis 114, resulting in the formation of two genetically identical daughter cells 222 that can exit the cell cycle or reenter a subsequent cell cycle process 224.

Many cancerous cells are highly metabolically active and have high mitotic rates associated with cellular division. The methods and medical devices for treating cancer described herein can target mitosis in the rapidly dividing cancer cells.

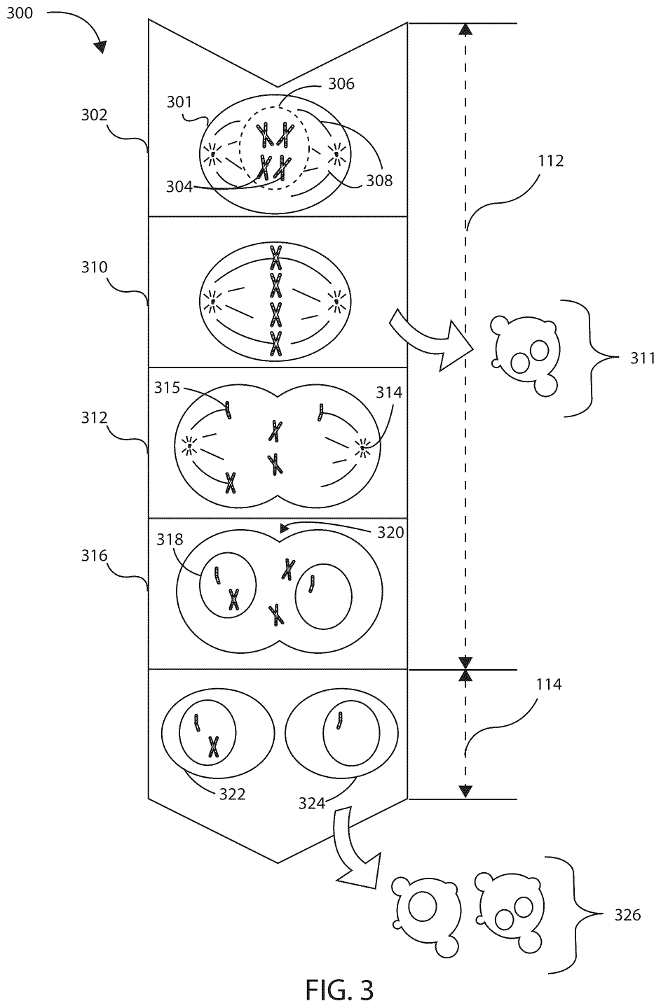

Referring now to FIG. 3, a schematic view of the M phase 300 in an exemplary cancerous cell 301 that has been disrupted by a chemotherapeutic agent and/or applied antimitotic therapy is shown in accordance with the embodiments herein. Mitosis within cancerous cell 301 begins similar to a healthy exemplary cell 200 as shown in FIG. 2. The process of mitosis within cancerous cell 301 begins in prophase 302, where the chromosomes 304 of the cancerous cell 301 begin to condense, the nuclear envelope 306 surrounding the nucleus disappears, and the spindle apparatus, including the spindle fibers 308, begin to form. An antimitotic agent and/or applied antimitotic therapy administered to cancerous cell 301 can destabilize spindle fiber 308 formation so that during metaphase 310, not all of the chromosomes become bound by the spindle fibers 308. In some embodiments, the chemotherapeutic agent is an antimitotic agent.

A consequence of destabilized spindle fibers can include mitotic arrest, or delay, in mitosis, which can lead to cell death (i.e., apoptosis) or mitotic slippage 311. A dividing cancerous cell can also proceed through mitosis through abnormal cellular division. If mitosis continues through abnormal cellular division and the chromosomes 304 cannot be separated evenly, then sister chromatids 315 and/or duplicated chromosomes 304 can be pulled towards the centrosomes 314 to opposite poles of the dividing cell and become unevenly distributed during anaphase 312. The cell can then proceed to telophase 316 where a nuclear membrane 318 can reform around each set of the chromosomes 304 at the opposite poles of the dividing cell, and a cleavage furrow 320 can form between the two halves of the cell. The final step in the cell cycle for the cancerous cell 301 is the step of cytokinesis 114, resulting in the formation of a first genetically distinct daughter cell 322 and a second genetically distinct daughter cell 324. In some embodiments, the genetically distinct daughter cells can die via apoptosis, reenter interphase of a subsequent cell cycle and die, or reenter mitosis 326.

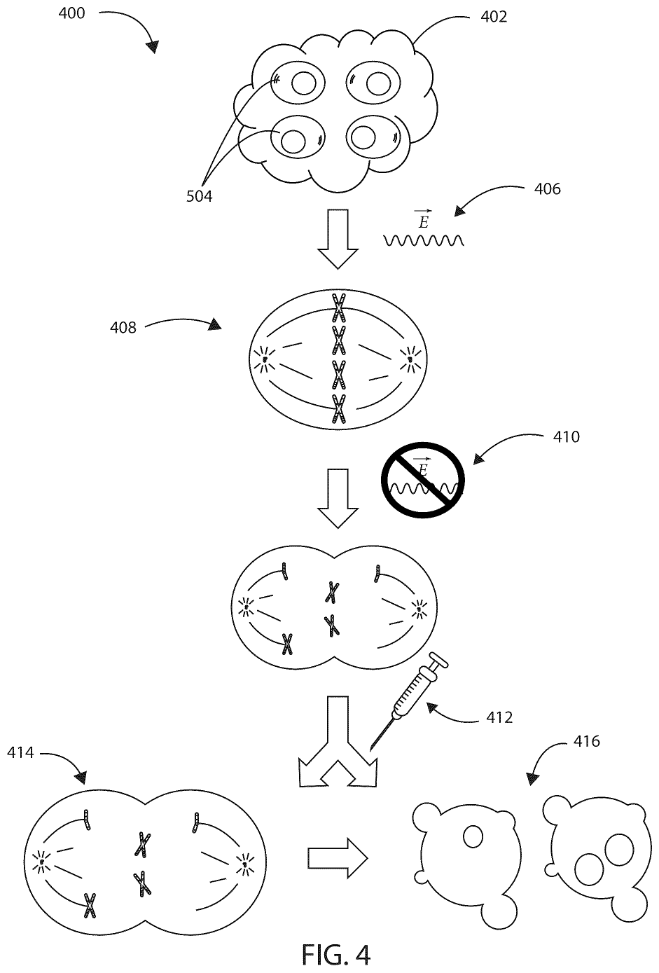

An exemplary method of treating cancer herein can include application of one or more TTF electric fields at or near the site of a cancerous tumor, tumor excision site, or targeted treatment site along with the administration of a chemotherapeutic agent. The administration of the TTF electrical fields can be before, after, and/or simultaneous with the administration of the chemotherapeutic agent. Referring now to FIG. 4, a schematic flow diagram of an exemplary method 400 for treating a cancerous tumor 402 located in a subject is shown in accordance with various embodiments herein. The method 400 can include applying one or more TTF electrical fields 406 at or near a site of the cancerous tumor 402. The cancerous tumor 402 can include a cancerous cell population 404. The one or more applied TTF electrical fields can be effective to delay mitosis in the cancerous cell population 404 and cause mitotic synchronization within at least a proportion of the cells within the cancerous cell population 408. The method 400 can include removing the one or more TTF electrical fields to allow mitosis to proceed within the cancerous cell population 410. The method 400 can include administering a chemotherapeutic agent at or near a site of the cancerous tumor after the one or more TTF electrical fields have been removed 412. In some embodiments, the chemotherapeutic agent can be delivered systemically through an intravenous port external to the body, or via an implantable device having an implantable conduit implanted within in the systemic vasculature, such as one implanted in the pectoral space. Administration of the chemotherapeutic agent can cause a disruption of mitosis within the cancerous cell population 414 and eventually lead to cell death within the cancerous cell population 416. In some embodiments, the method 400 can include inserting a transcutaneous access port at or near the site of the cancerous tumor.

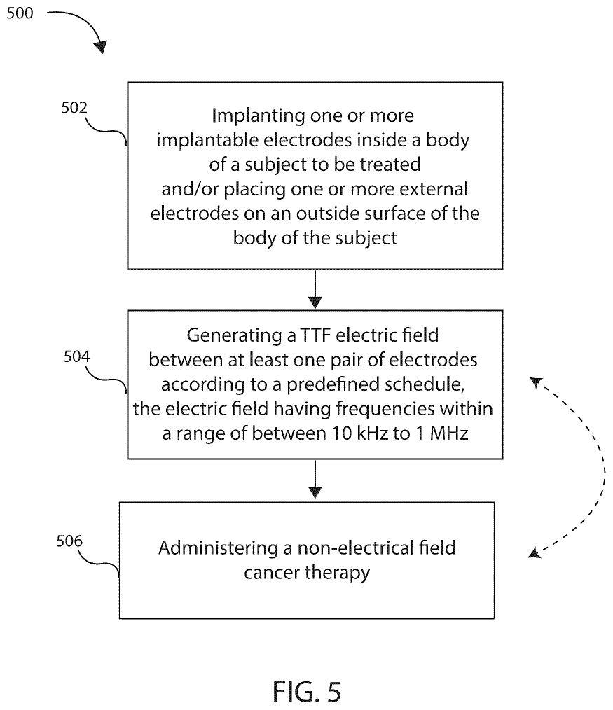

The methods herein can include the use of one or more implantable electrodes to treat a cancerous tumor, tumor excision site, or targeted treatment site. Referring now to FIG. 5, a method 500 for treating cancer is shown in accordance with various methods herein. The method 500 includes implanting one or more implantable electrodes inside a body of a subject to be treated and/or placing one or more external electrodes on an outside surface of the body of the subject 502. The method includes generating a TTF electrical field between at least one pair of electrodes 504, the TTF electrical field having frequencies within a range of between 10 kHz to 1 MHz. The method 500 also includes administering a non-electrical field cancer therapy (examples described herein) at or near a site of the cancerous tumor, or systemically 506. As indicated, the TTF electrical field therapy 504 can occur before or after the non-electrical field therapy 506 and/or the different types of therapy can occur simultaneously.

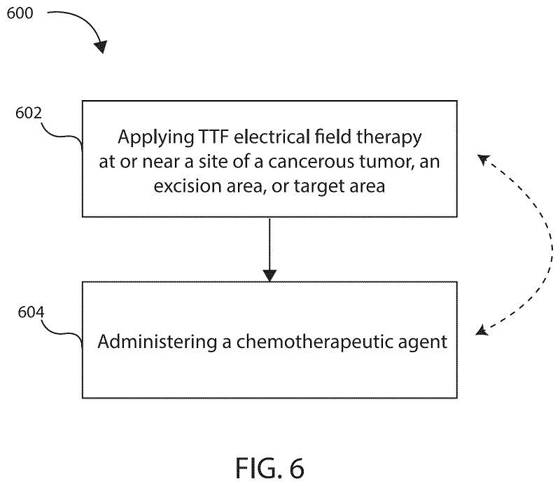

Referring now to FIG. 6, a method 600 for treating cancer is shown with various embodiments herein. The method 600 can include applying a TTF electrical field at or near a site of the cancerous tumor, tumor excision site, or targeted treatment site 602. The method 600 can also include administering a chemotherapeutic agent systemically and/or at or near a site of the cancerous tumor, tumor excision site, or targeted treatment site after the first TTF electrical field has been removed 604. As indicated, the TTF electrical field therapy 602 can occur before or after the chemotherapy 604 and/or the different types of therapy can occur simultaneously.

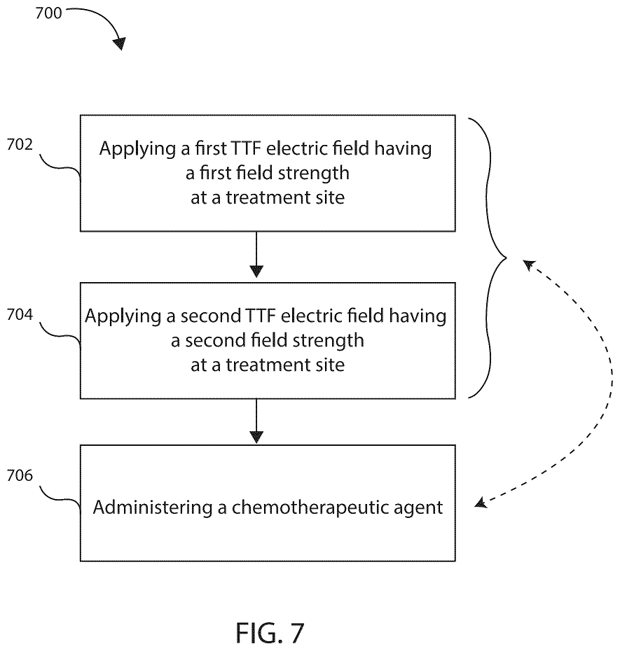

Referring now to FIG. 7, a method 700 for treating cancer is shown with various embodiments herein. The method 700 can include applying a first TTF electrical field having a first field strength at or near a site of the cancerous tumor, tumor excision site, or targeted treatment site 702 across a vector resultant from selected electrodes. The method 700 can include applying a second TTF electrical field having a second TTF electrical field strength at or near the site of the cancerous tumor after removing the first TTF electrical field and prior to administering the chemotherapeutic agent 706. The method 700 can include administering a chemotherapeutic agent 706 systemically and/or at or near a site of the cancerous tumor, tumor excision site, or targeted treatment site. In some embodiments, the method 700 can include applying a third TTF electrical field having a third field strength at or near the site of a cancerous tumor. In some embodiments, the method 700 can include applying a fourth TTF electrical field having a fourth field strength at or near the site of a cancerous tumor. In other embodiments, the method 700 can include applying a fifth, sixth, seventh, eighth, ninth, or tenth TTF electrical field at or near the site of a cancerous tumor. As indicated, the TTF electrical field therapy can occur before, after, or simultaneously with the chemotherapy.

In some embodiments, the methods of applying a second or greater TTF electrical field having a second or greater electric field strength can include waiting a predetermined amount of time between applications of successive TTF electrical fields. By way of example, the method 700 can include waiting a predetermined amount of time after removing the first TTF electrical field prior to applying the second TTF electrical field. Similarly, in the application of a third TTF electrical field having a third TTF electrical field strength, application of the third TTF electrical field can be delayed by waiting a predetermined amount of time after removing the second TTF electrical field prior to applying the third TTF electrical field. However, in some embodiments, applying a second or greater TTF electrical field having a second or greater electric field strength can include applying the second or greater TTF electrical field immediately after application of the preceding TTF electrical field.

In some embodiments, the second TTF electrical field strength is less than the first TTF electrical field strength. In some embodiments, the second TTF electrical field strength is greater than the first TTF electrical field strength. In other embodiments, the second TTF electrical field strength is the same as the first TTF electrical field strength. In some embodiments, each successive application of an additional TTF electrical field having its unique electric field strength can include the additional TTF electrical field having an TTF electrical field strength that is less than, that is greater than, or that is the same as the preceding or successive TTF electrical fields.

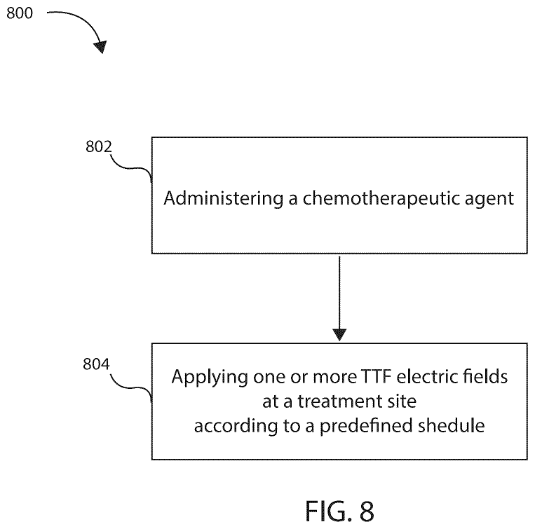

Application of the one or more TTF electrical fields in the methods herein can be temporally controlled. Referring now to FIG. 8, a method 800 for treating cancer is shown in accordance with the embodiments herein. The method 800 can include administering a chemotherapeutic agent at or near a site of the cancerous tumor, tumor excision site, or targeted treatment site 802. The method 800 can also include applying one or more TTF electrical fields at or near a site of the cancerous tumor, tumor excision site, or targeted treatment site according to a predefined schedule 804. The predefined schedule can cause the TTF electrical fields to vary in at least one of intensity and frequency over the course of a defined time period of at least six hours.

Temporal control of the application of the one or more TTF electrical fields can include temporal variation of at least one of the intensity and frequency of the one or more TTF electrical fields on a predefined schedule. In some embodiments, temporal control of the application of the one or more TTF electrical fields can include temporal variation of at least one of the intensity and frequency, as compared to an initial intensity of frequency, of the one or more TTF electrical fields on a predefined schedule. In some embodiments, the predefined schedule includes one or more predetermined down periods wherein the one or more applied TTF electrical fields is decreased in intensity or frequency by at least 50% for at least 4 hours. In some embodiments, the predefined schedule includes one or more predetermined down periods wherein the one or more applied TTF electrical fields is decreased in intensity or frequency by at least 75% for at least 4 hours. In some embodiments, the predefined schedule includes one or more predetermined down periods wherein the one or more applied TTF electrical fields is decreased in intensity or frequency by greater than or equal to 25%, 30%, 35%, 40%, 45%, 50%, 55%, 60%, 65%, 70%, 75%, 80%, 85%, 90%, 95%, 98%, 99% or 100% or can be an amount falling in a range within any of the foregoing.

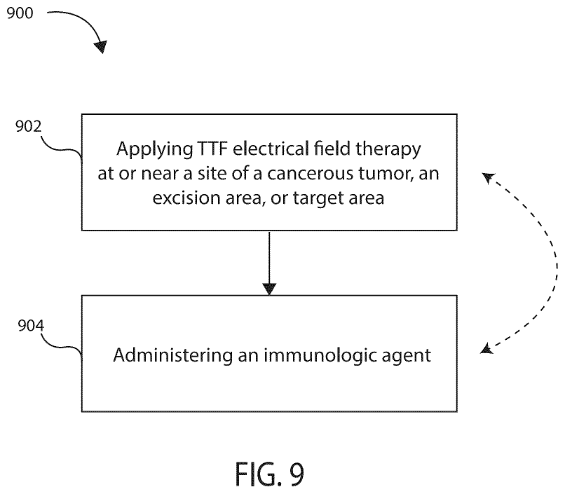

In some embodiments, a method 900 for treating cancer is included. FIG. 9 shows a flow chart showing a method 900 of treating cancer by administering electrical therapy and one or more immunologic agents according to an embodiment.

In some embodiments, the method 900 can include applying a first TTF electrical field having a first field strength at or near a site of the cancerous tumor, tumor excision site, or targeted treatment site 902.

In various embodiments, the method 900 can include administering an immunologic agent at or near a site of the cancerous tumor, tumor excision site, or targeted treatment site 904. In some embodiments, the immunologic agents can be administered through an intravenous port external to the body, through ports fully under the skin with or without the need to access a vein directly, through subcutaneous injection beneath the skin, and/or through other modalities. In some embodiments, administration of the immunologic agent can work to activate the immune system to recognize and attack cancerous cell populations. In other embodiments, administration of the immunologic agent can work to target specific proteins or molecules on cancerous cells discussed in greater detail below.

As indicated, the TTF electrical field therapy 902 can occur before or after administering an immunologic agent systemically and/or at or near a site of the cancerous tumor, tumor excision site, or targeted treatment site 904 and/or the different types of therapy can occur simultaneously.

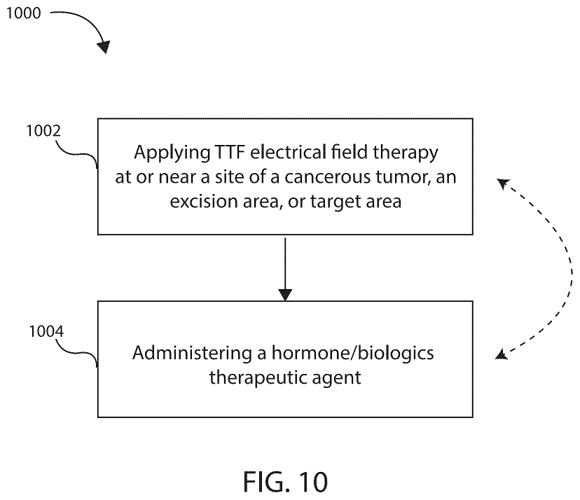

In some embodiments, a method 1000 for treating cancer located within a subject is included. FIG. 10 shows a flow chart showing a method 1000 of treating cancer by administering electrical therapy and one or more hormone/biologic therapeutic agents according to an embodiment.

In some embodiments, the method 1000 can include applying a first TTF electrical field having a first field strength at or near a site of the cancerous tumor, tumor excision site, or targeted treatment site 1002.

In various embodiments, the method 1000 can include administering a hormone/biologic therapeutic agent systemically and/or at or near a site of the cancerous tumor, tumor excision site, or targeted treatment site 1004. In some embodiments, the hormone/biologic therapeutic agents can be delivered systemically through an intravenous port external to the body, or via an implantable device having an implantable conduit implanted within in the systemic vasculature, such as one implanted in the pectoral space. In some embodiments, administration of the hormone/biologic therapeutic agent can work to target hormones or hormone receptors to slow or stop the growth of the cancerous cells.

As indicated, the TTF electrical field therapy 1002 can occur before or after administering a hormone/biologic therapeutic agent 1004 and/or the different types of therapy can occur simultaneously.

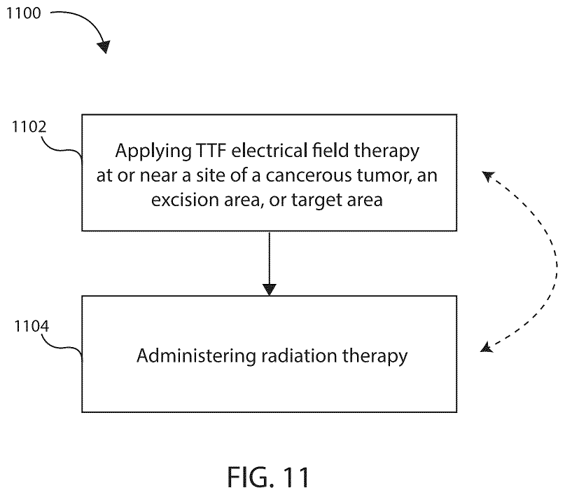

In some embodiments, a method 1100 for treating cancer located within a subject is included. FIG. 11 shows a flow chart showing a method 1100 of treating cancer by administering electrical therapy and radiation therapy according to an embodiment.

In some embodiments, the method 1100 can include applying a TTF electrical field at or near a site of the cancerous tumor, tumor excision site, or targeted treatment site 1102.

In various embodiments, the method 1100 can include administering radiation therapy at or near a site of the cancerous tumor, tumor excision site, or targeted treatment site 1104. In some embodiments, radiation therapy can be delivered external to the body by delivering radiation beams at or near a cancerous site, or internally by delivering at or near to the cancerous site radioactive material. In some embodiments, radiation therapy can use radiation to damage or destroy cancerous cells by damaging its DNA.

As indicated, the TTF electrical field therapy 1102 can occur before, after, or even during the administration of radiation therapy 1104.

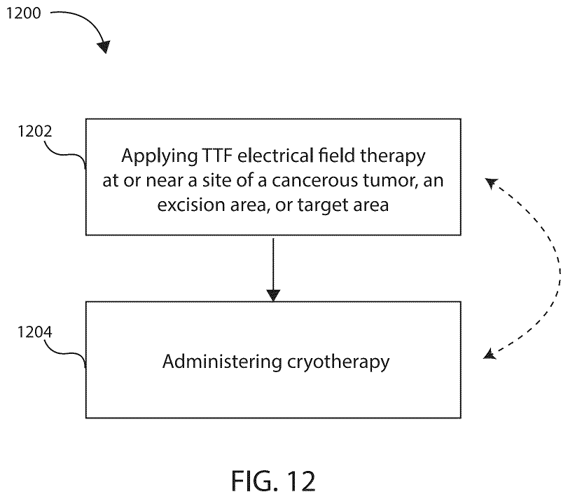

In some embodiments, a method 1200 for treating cancer is included. FIG. 12 shows a flow chart showing a method 1200 of treating cancer by administering electrical therapy and cryotherapy according to an embodiment.

In some embodiments, the method 1200 can include applying a TTF electrical field at or near a site of the cancerous tumor, tumor excision site, or targeted treatment site 1202.

In various embodiments, the method 1200 can include administering cryotherapy at or near a site of the cancerous tumor, tumor excision site, or targeted treatment site 1204. In some embodiments, cryotherapy can be delivered external to the body by delivering at or near to a cancerous site cool, subzero, temperatures, or internally by delivering at or near to the cancerous site cool, subzero, temperatures. In some embodiments, cryotherapy can use cool, subzero, temperatures to freeze or destroy cancerous cells.

As indicated, the TTF electrical field therapy 1202 can occur before or after administering cryotherapy 1204 and/or the different types of therapy can occur simultaneously.

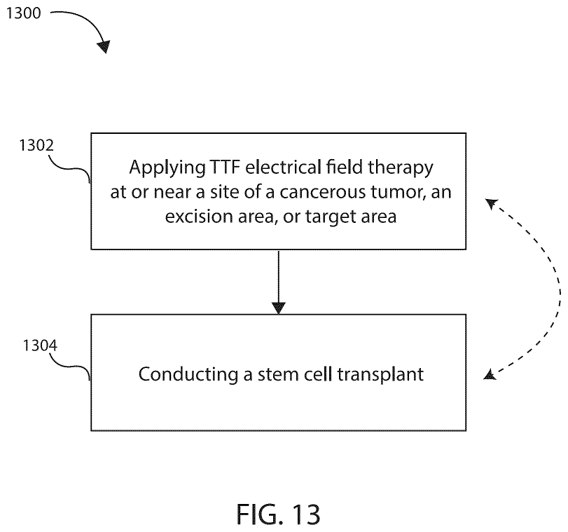

In some embodiments, a method 1300 for treating cancer is included. FIG. 13 shows a flow chart showing a method 1300 of treating cancer by administering electrical therapy and conducting a stem cell transplant according to an embodiment.

In some embodiments, the method 1300 can include applying a TTF electrical field at or near a site of the cancerous tumor, tumor excision site, or targeted treatment site 1302.

In various embodiments, the method 1300 can include conducting a stem cell transplant 1304. In some embodiments, the stem cell transplant can replace the diseased or damaged bone marrow or blood cells with healthy stem cells to help restore the patient's ability to produce normal blood cells and immune cells.

As indicated, the TTF electrical field therapy 1302 can occur before or after conducting a stem cell transplant 1304 and/or the different types of therapy can occur simultaneously.

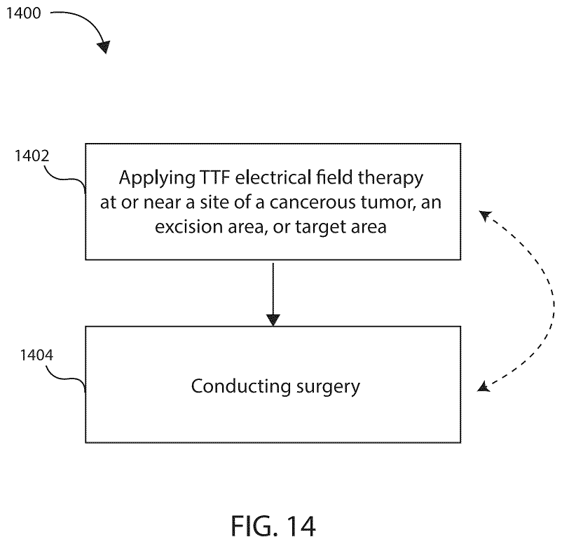

In some embodiments, a method 1400 for treating cancer is included. FIG. 14 shows a flow chart showing a method 1400 of treating a cancerous tumor by administering electrical therapy and conducting surgery according to an embodiment.

In some embodiments, the method 1400 can include applying a TTF electrical field at or near a site of the cancerous tumor 1402. In various embodiments, the method 1400 can include conducting surgery 1404. In some embodiments, surgery can partially or completely remove cancerous tissue. As indicated, the TTF electrical field therapy 1402 can occur before or after conducting surgery 1404 and/or the different types of therapy can occur simultaneously.

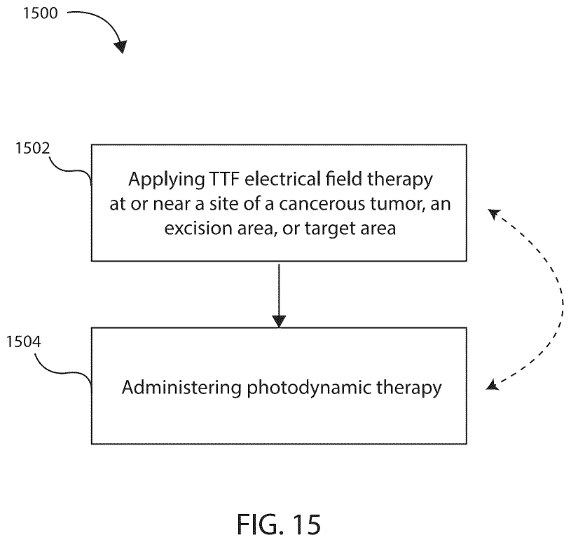

In some embodiments, a method 1500 for treating cancer is included. FIG. 15 shows a flow chart illustrating a method 1500 of treating cancer by administering electrical therapy and photodynamic therapy according to an embodiment.

In some embodiments, the method 1500 can include applying a TTF electrical field at or near a site of the cancerous tumor, tumor excision site, or targeted treatment site 1502.

In various embodiments, the method 1500 can include conducting photodynamic therapy 1504. In some embodiments, the photodynamic therapy be conducted external to the body by removing a patient's blood and treating the blood before returning the blood to the patient, or internally by treating the patient's blood within his/her body. In some embodiments, photodynamic therapy can selectively destroy cancerous cells. As indicated, the TTF electrical field therapy 1502 can occur before or after conducting photodynamic therapy 1504 and/or the different types of therapy can occur simultaneously.

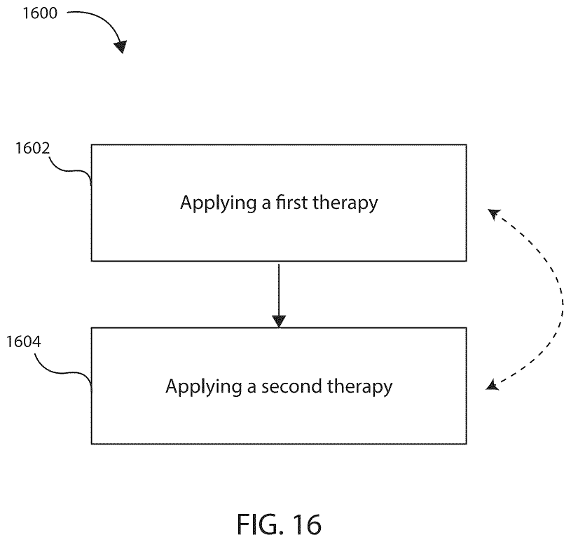

Referring now to FIG. 16, shows a flow chart showing a method 1600 of treating cancer by initiating a first therapy 1602. The first therapy 1602 can include, but is not limited to, electrical therapy, chemotherapy, cryotherapy, surgery, immunotherapy, photodynamic therapy, stem cell transplant therapy, radiation therapy, telomerase inhibition therapy, gene therapy, hormone/biological therapy, and/or any other therapies described herein.

In various embodiments, the method 1600 can include initiating a second therapy 1604. The second therapy 1604 can include, but is not limited to, electrical therapy, chemotherapy, cryotherapy, surgery, immunotherapy, photodynamic therapy, stem cell transplant therapy, radiation therapy, gene therapy, telomerase inhibition therapy, hormone/biological therapy, and/or any other therapies described herein.

In some embodiments, the first therapy and the second therapy can be initiated simultaneously. In various embodiments, multiple rounds of the first therapy and the second therapy can be initiated simultaneously. For example, 1-60 rounds of the first therapy and the second therapy can be initiated. In some embodiments, 1, 5, 10, 15, 20, 25, 30, 35, 40, 45, 50, 55, 60, 120, 180, 360, 720, or more rounds, or a number of rounds falling in a range within any of the foregoing numbers of rounds of the first therapy and the second therapy can be initiated. As described herein, a round refers to one of a sequence of sessions of a therapy, where each session is considered a therapeutically effective dose.

In other embodiments, the first therapy can be conducted before the second therapy. In various embodiments, multiple rounds of the first therapy can be initiated prior to the second therapy. For example, 1 to 60 rounds of the first therapy can be initiated prior to the initiation of the second therapy. In some embodiments, 1, 5, 10, 15, 20, 25, 30, 35, 40, 45, 50, 55, or 60 or can be an amount falling in a range within any of the foregoing rounds of the first therapy can be initiated prior to the initiation of the second therapy. In other embodiments, the second therapy can be initiated between rounds of the first therapy. In some embodiments, the second therapy can be initiated between rounds of the first therapy and after all rounds of the first therapy.

In various embodiments, multiple rounds of the second therapy can be initiated after the initiation of the one or more rounds of the first therapy. For example, 1 to 60 rounds of the second therapy can be initiated after the initiation of the first therapy. In some embodiments, 1, 5, 10, 15, 20, 25, 30, 35, 40, 45, 50, 55, or 60 or can be an amount falling in a range within any of the foregoing rounds of the second therapy can be initiated. In some embodiments, the first therapy can be re-initiated after all rounds of the second therapy.

In some embodiments, the first therapy and the second therapy can be the same therapy. For example, the first therapy and the second therapy can both be electrical therapies. In other embodiments, the first therapy and the second therapy can be different therapies. For example, the first therapy can be surgery and the second therapy can be electrical therapy.

In some embodiments, one or more rounds of the first therapy can be initiated for a matter of minutes, hours, days, weeks, or months. For example, one or more rounds of the first therapy can be initiated for 1 minute, 4 hours, 6 days, 8 weeks, or 10 months, or can be an amount falling in a range within any of the foregoing. In some embodiments, one or more rounds of the second therapy can be initiated for a matter of minutes, hours, days, weeks, or months. For example, one or more rounds of the second therapy can be initiated for 1 minute, 4 hours, 6 days, 8 weeks, or 10 months, or can be an amount falling in a range within any of the foregoing.

It is further contemplated herein that a third therapy, fourth therapy, fifth therapy, and/or sixth therapy can be initiated at any point before, simultaneously, and/or after the first therapy and the second therapy.

In the various methods described herein, applying the one or more TTF electrical fields can include at least applying an electric field at various electric field strengths. By way of example, the one or more TTF electrical fields can be applied to the cancerous tumor, tumor excision site, or targeted treatment site at electric field strengths selected from a range of electric field strengths from 0.25 V/cm to 50 V/cm. In some embodiments, the one or more electric fields can be applied to the cancerous tumor, tumor excision site, or targeted treatment site at electric field strengths selected from a range of electric field strengths from 1 V/cm to 10 V/cm. In some embodiments, the one or more electric fields can be applied to the cancerous tumor, tumor excision site, or targeted treatment site at electric field strengths selected from a range of electric field strengths from 1 V/cm to 5 V/cm. In some embodiments, the one or more electric fields can be applied to the cancerous tumor, tumor excision site, or targeted treatment site at electric field strengths selected from a range of electric field strengths from 3 V/cm to 5 V/cm. In some embodiments, the field strength can be greater than or equal to 0.25 V/cm, 0.50 V/cm, 0.75 V/cm, 1.00 V/cm, 1.25 V/cm, 1.50 V/cm, 1.75 V/cm, 2.00 V/cm, 2.25 V/cm, 2.50 V/cm, 2.75 V/cm, 3.00 V/cm, 3.25 V/cm, 3.50 V/cm, 3.75 V/cm, 4.00 V/cm, 4.25 V/cm, 4.50 V/cm, 4.75 V/cm, 5.00 V/cm, 5.25 V/cm, 5.50 V/cm, 5.75 V/cm, 6.00 V/cm, 6.25 V/cm, 6.50 V/cm, 6.75 V/cm, 7.00 V/cm, 7.25 V/cm, 7.50 V/cm, 7.75 V/cm, 8.00 V/cm, 8.25 V/cm, 8.50 V/cm, 8.75 V/cm, 9.00 V/cm, 9.25 V/cm, 9.50 V/cm, 9.75 V/cm, 10 V/cm, 20 V/cm, 30 V/cm, 40 V/cm or 50 V/cm, or can be an amount falling in a range within any of the foregoing.

Field strengths used herein can be sufficient to disrupt mitosis within cancerous cells and yet not be strong enough to cause other effects such as electroporation. Thus, the application of TTF electrical fields herein does not encompass electrical fields used for electroporation.

A desired electric field strength can be achieved by delivering an electric current between two electrodes. The specific current and voltage at which the TTF electrical field is delivered can vary and can be adjusted to achieve the desired electric field strength at the site of the tissue to be treated. In some embodiments, control circuitry can be configured to direct a TTF electrical field generating circuit to deliver an electric field using currents ranging from 1 mAmp to 1000 mAmp to the site of a cancerous tumor, tumor excision site, or targeted treatment site. In some embodiments, the control circuitry can be configured to direct the TTF electrical field generating circuit to deliver an electric field using currents ranging from 20 mAmp to 500 mAmp to the site of a cancerous tumor, tumor excision site, or targeted treatment site. In some embodiments, the control circuitry can be configured to direct the TTF electrical field generating circuit to deliver an electric field using currents ranging from 30 mAmp to 300 mAmp to the site of a cancerous tumor, tumor excision site, or targeted treatment site.

In some embodiments, the control circuitry can be configured to direct the TTF electrical field generating circuit to deliver an electric field using currents including 1 mAmp, 2 mAmp, 3 mAmp, 4 mAmp, 5 mAmp, 6 mAmp, 7 mAmp, 8 mAmp, 9 mAmp, 10 mAmp, 15 mAmp, 20 mAmp, 25 mAmp, 30 mAmp, 35 mAmp, 40 mAmp, 45 mAmp, 50 mAmp, 60 mAmp, 70 mAmp, 80 mAmp, 90 mAmp, 100 mAmp, 125 mAmp, 150 mAmp, 175 mAmp, 200 mAmp, 225 mAmp, 250 mAmp, 275 mAmp, 300 mAmp, 325 mAmp, 350 mAmp, 375 mAmp, 400 mAmp, 425 mAmp, 450 mAmp, 475 mAmp, 500 mAmp, 525 mAmp, 550 mAmp, 575 mAmp, 600 mAmp, 625 mAmp, 650 mAmp, 675 mAmp, 700 mAmp, 725 mAmp, 750 mAmp, 775 mAmp, 800 mAmp, 825 mAmp, 850 mAmp, 875 mAmp, 900 mAmp, 925 mAmp, 950 mAmp, 975 mAmp, or 1000 mAmp. It will be appreciated that the control circuitry can be configured to direct the electric field generating circuit to deliver an electric field at a current falling within a range, wherein any of the forgoing currents can serve as the lower or upper bound of the range, provided that the lower bound of the range is a value less than the upper bound of the range.

In some embodiments, the control circuitry can be configured to direct the TTF electrical field generating circuit to deliver an electric field using voltages ranging from 1 Vᵣₘₛ to 50 Vᵣₘₛ to the site of a cancerous tumor, tumor excision site, or targeted treatment site. In some embodiments, the control circuitry can be configured to direct the TTF electrical field generating circuit to deliver an electric field using voltages ranging from 5 Vᵣₘₛ to 30 Vᵣₘₛ to the site of a cancerous tumor, tumor excision site, or targeted treatment site. In some embodiments, the control circuitry can be configured to direct the TTF electrical field generating circuit to deliver an TTF electrical field using voltages ranging from 10 Vᵣₘₛ to 20 Vᵣₘₛ to the site of a cancerous tumor, tumor excision site, or targeted treatment site.

In some embodiments, the control circuitry can be configured to direct the TTF electrical field generating circuit to deliver an electric field using one or more voltages including 1 Vᵣₘₛ, 2 Vᵣₘₛ, 3 Vᵣₘₛ, 4 Vᵣₘₛ, 5 Vᵣₘₛ, 6 Vᵣₘₛ, 7 Vᵣₘₛ, 8 Vᵣₘₛ, 9 Vᵣₘₛ, 10 Vᵣₘₛ, 15 Vᵣₘₛ, 20 Vᵣₘₛ, 25 Vᵣₘₛ, 30 Vᵣₘₛ, 35 Vᵣₘₛ, 40 Vᵣₘₛ, 45 Vᵣₘₛ, or 50 Vᵣₘₛ. It will be appreciated that the control circuitry can be configured to direct the TTF electrical field generating circuit to deliver an electric field using a voltage falling within a range, wherein any of the forgoing voltages can serve as the lower or upper bound of the range, provided that the lower bound of the range is a value less than the upper bound of the range.

In the various methods described herein, applying the one or more TTF electrical fields can include at least applying an electric field at various frequencies. The one or more TTF electrical fields can be applied to the cancerous tumor, tumor excision site, or targeted treatment site at frequencies selected from a range within 10 kilohertz (kHz) to 1 megahertz (MHz). In some embodiments, the one or more TTF electrical fields can be applied to the cancerous tumor, tumor excision site, or targeted treatment site at frequencies selected from a range within 100 kHz to 500 kHz. In some embodiments, the one or more TTF electrical fields can be applied to the cancerous tumor, tumor excision site, or targeted treatment site at frequencies selected from a range within 100 kHz to 300 kHz. In some embodiments, the frequency of the one or more applied TTF electrical fields can be greater than or equal to 10 kHz, 20 kHz, 30 kHz, 40 kHz, 50 kHz, 60 kHz, 70 kHz, 80 kHz, 90 kHz, 100 kHz, 125 kHz, 150 kHz, 175 kHz, 200 kHz, 225 kHz, 250 kHz, 275 kHz, 300 kHz, 325 kHz, 350 kHz, 375 kHz, 400 kHz, 425 kHz, 450 kHz, 475 kHz, 500 kHz, 525 kHz, 550 kHz, 575 kHz, 600 kHz, 625 kHz, 650 kHz, 675 kHz, 700 kHz, 725 kHz, 750 kHz, 775 kHz, 800 kHz, 825 kHz, 850 kHz, 875 kHz, 900 kHz, 925 kHz, 950 kHz, 975 kHz, or 1 MHz or can be an amount falling in a range within any of the foregoing.

In various embodiments herein, the TTF electrical field can be released (ceased) and then a chemotherapeutic agent can be administered. In various embodiments, the amount of time between releasing the TTF electrical field and administering the chemotherapeutic agent can be about 0, 5, 10, 15, 20, 25, 30, 40, 50, 60, 90, 120, or 180 minutes, of an amount falling within a range between any of the foregoing.

In the various methods described herein, applying the one or more TTF electrical fields can include at least applying a TTF electrical field for various predetermined time periods. The one or more TTF electrical fields can be applied at or near the site of the cancerous tumor, tumor excision site, or targeted treatment site over a predetermined time period selected from a range of predetermined time periods from 1 minute to 24 hours. In some embodiments, the one or more TTF electrical fields can be applied at or near the site of the cancerous tumor, tumor excision site, or targeted treatment site over a predetermined time period can be greater than or equal to 1, 10, 20, 30, 40, or 50 minutes, or 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.0, 20.5, 21.0, 21.5, 22.0, 22.5, 23.0, 23.5, 24.0, or 48 hours, or can be an amount falling in a range within any of the foregoing.

In the various methods described herein, administering a chemotherapeutic agent can include administering the chemotherapeutic agent when at least a certain percentage of the population is synchronized in mitosis. In some embodiments, administering the chemotherapeutic agent to the cancerous tumor includes administering the chemotherapeutic agent when at least 5% of the cancerous cell population is synchronized in mitosis in response to the one or more TTF electrical fields. In some embodiments, administering the chemotherapeutic agent to the cancerous tumor includes administering the chemotherapeutic agent when at least 25% of the cancerous cell population is synchronized in mitosis in response to the one or more TTF electrical fields. In some embodiments, administering the chemotherapeutic agent to the cancerous tumor includes administering the chemotherapeutic agent when at least 50% of the cancerous cell population is synchronized in mitosis in response to the one or more TTF electrical fields. In some embodiments, administering the chemotherapeutic agent to the cancerous tumor includes administering the chemotherapeutic agent when at least 75% of the cancerous cell population is synchronized in mitosis in response to the one or more TTF electrical fields. In some embodiments, the percentage of cells in a state of delayed mitosis and mitotic synchronization can be greater than or equal to 5%, 10%, 15%, 20%, 25%, 30%, 35%, 40%, 45%, 50%, 55%, 60%, 65%, 70%, 75%, 80%, 85%, 90%, or 95%, or can be an amount falling in a range within any of the foregoing.

In the various methods described herein, applying the one or more TTF electrical fields at or near the site of the cancerous tumor can include applying the one or more TTF electrical fields from the exterior or interior of the subject (e.g., using external electrodes or implanted electrodes). In some embodiments, applying the one or more TTF electrical fields to the cancerous tumor can include applying the one or more TTF electrical fields entirely from the exterior of the subject using external electrodes at or near the site of the cancerous tumor. In some embodiments, applying the one or more TTF electrical fields to the cancerous tumor can include applying the one or more TTF electrical fields entirely from the interior of the subject using implanted electrodes at or near the site of the cancerous tumor. In some embodiments, applying the one or more TTF electrical fields to the cancerous tumor can include applying the one or more TTF electrical fields at least partially from the exterior of the subject at or near the site of the cancerous tumor. In some embodiments, applying the one or more TTF electrical fields to the cancerous tumor can include applying the one or more TTF electrical fields at least partially from the interior of the subject at or near the site of the cancerous tumor. In other embodiments, applying the one or more TTF electrical fields to the cancerous tumor can include applying the one or more TTF electrical fields partially from the interior and partially from the exterior of the subject at or near the site of the cancerous tumor. It will be appreciated that applying a TTF electrical field from the exterior of a subject can result in propagation of the electric field into the body of the subject.

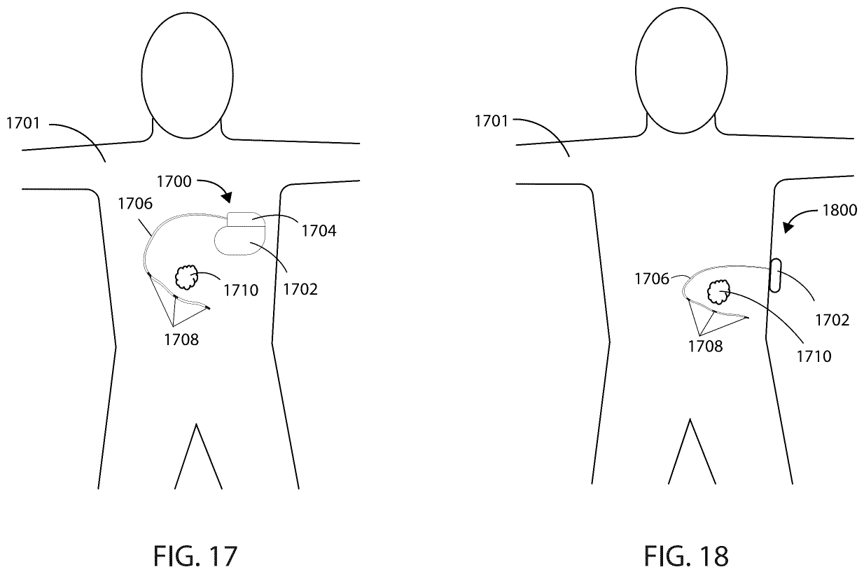

Therapies herein can be delivered to a subject with a cancer using a variety of medical devices. Referring now to FIG. 17 and FIG. 18, schematic diagrams of a subject 1701 with a cancerous tumor 1710 are shown in accordance with embodiments herein. In FIG. 17, the subject 1701 has a medical device 1700 implanted entirely within the body of the subject 1701 at or near the site of a cancerous tumor located within a bodily tissue. Various implant sites can be used including areas such as in the limbs, the upper torso, the abdominal area, the head, and the like. In FIG. 18, the subject 1701 has a medical device 1700 at least partially implanted within body of the subject 1701 at or near the site of a cancerous tumor located within a bodily tissue. In some embodiments, the medical device can be entirely external to the subject. In some embodiments, the medical device can be partially external to the subject. In some embodiments, the medical device can be partially implanted and partially external to the body of a subject. In other embodiments, a partially implanted medical device can include a transcutaneous connection between components disposed internal to the body and external to the body. A partially implanted medical device can wirelessly communicate with a partially external portion of a medical device over a wireless connection.

In some embodiments, a portion of the medical device can be entirely implanted and a portion of the medical device can be entirely external. For example, in some embodiments, one or more electrodes or leads can be entirely implanted within the body, whereas the portion of the medical device that generates a TTF electrical field, such as an electric field generator, can be entirely external to the body. It will be appreciated that in some embodiments described herein, the electric field generators described can include the many of the same components as and can be configured to perform many of the same functions as a pulse generator. In embodiments where a portion of a medical device is entirely implanted, and a portion of the medical device is entirely external, the portion of the medical device that is entirely external can communicate wirelessly with the portion of the medical device that is entirely internal. However, in other embodiments a wired connection can be used.

The medical device 1700 can include a housing 1702 and a header 1704 coupled to the housing 1702, and medical device 1700 can include a housing 1702. Various materials can be used. However, in some embodiments, the housing 1702 can be formed of a material such as a metal, ceramic, polymer, composite, or the like. In some embodiments, the housing 1702, or one or more portions thereof, can be formed of titanium. The header 1704 can be formed of various materials, but in some embodiments the header 1704 can be formed of a translucent polymer such as an epoxy material. In some embodiments the header 1704 can be hollow. In other embodiments the header 1704 can be filled with components and/or structural materials such as epoxy or another material such that it is non-hollow.

In some embodiments where a portion of the medical device 1700 or 1800 is partially external, the header 1704 and housing 1702 can be surrounded by a protective casing made of durable polymeric material. In other embodiments, where a portion of the medical device 1700 or 1800 is partially external, the header 1704 and housing 1702 can be surrounded by a protective casing made of a combination of polymeric material, metallic material, and/or glass material.

The header 1704 can be coupled to one or more leads 1706. The header 1704 can serve to provide fixation of the proximal end of one or more leads 1706 and electrically couple the one or more leads 1706 to one or more components within the housing 1702. The one or more leads 1706 can include one or more electrodes 1708 disposed along the length of the electrical leads 1706. In some embodiments, electrodes 1708 can include TTF electrical field generating electrodes and in other embodiments electrodes 1708 can include electric field sensing electrodes. In some embodiments, leads 1706 can include both electric field generating and electric field sensing electrodes. In other embodiments, leads 1706 can include any number of electrodes that are both electric field sensing and electric field generating. It will be appreciated that while many embodiments of medical devices herein are designed to function with leads, leadless medical devices that generate TTF electrical fields are also contemplated herein. In some embodiments, the electrodes 1708 can be tip electrodes on the most distal end of the leads 1706.

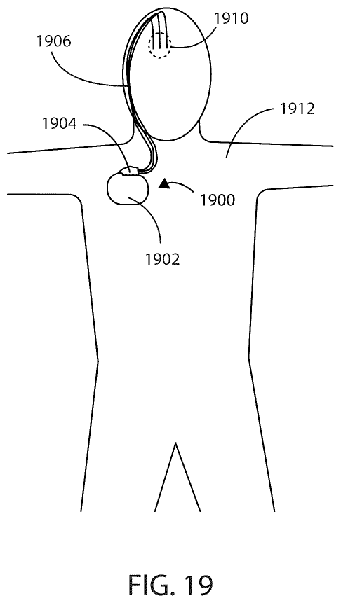

The therapies herein can be delivered to a subject/patient with a cancerous tumor resection site in their head. Referring now to FIG. 19, a schematic view of a medical device 1900 implanted in a patient 1912 is shown in accordance with the embodiments herein. In FIG. 19, the patient 1912 has the medical device 1900 implanted entirely within the body of the patient 1912 at or near a tumor resection site 1910. It will be appreciated that while many embodiments herein disclose a tumor resection site 1910, that area may alternatively represent a cancerous or non-cancerous tumor site, zone, or cavity. Various implant sites can be used including areas such as in the limbs, the upper torso, the abdominal area, the head, and the like. In some embodiments, the medical device can be at least partially implanted within the body of the patient at or near the site of the cancerous tumor.

The medical device 1900 can include a housing 1902 and a header 1904 coupled to the housing 1902. Various materials can be used. However, in some embodiments, the housing 1902 can be formed of a material such as a metal, ceramic, polymer, composite, or the like. In some embodiments, the housing 1902 and/or portions thereof can specifically be formed of a conductive material. In some embodiments, the housing 1902, or one or more portions thereof, can be formed of titanium. The header 1904 can be formed of various materials, but in some embodiments the header 1904 can be formed of a translucent polymer such as an epoxy material. In some embodiments the header 1904 can be hollow. In other embodiments the header 1904 can be filled with components and/or structural materials such as epoxy or another material such that it is non-hollow.

In some embodiments where a portion of the medical device 1900 is partially external, the header 1904 and housing 1902 can be surrounded by a protective casing made of durable polymeric material. In other embodiments, where a portion of the medical device 1900 is partially external, the header 1904 and housing 1902 can be surrounded by a protective casing made of a combination of polymeric material, metallic material, and/or glass material.

Header 1904 can be coupled to one or more leads, such as leads 1906. The header 1904 can serve to provide fixation of the proximal end of one or more leads 1906 and electrically couple the one or more leads 1906 to one or more components within the housing 1902. As such, one or more connection terminals, contacts, and/or pins can be disposed within the header 1904.

The one or more leads 1906 can include one or more electrodes (not shown in this view) disposed along the length of the leads 1906. In some embodiments, electrodes can include supply electrodes, also referred to herein as “TTF electrical field therapy supply electrodes.” In some embodiments electrodes can include electric field sensing electrodes, also referred to herein as “sensing electrodes.” In some embodiments, leads 1906 can include both supply electrodes and sensing electrodes. In other embodiments, leads 1906 can include any number of electrodes that are both supply electrodes and sensing electrodes.

The one or more leads 1906 can also include one or more temperature sensors (not shown in this view) disposed along the length of the leads 1906. Temperature sensors herein can include, but are not limited to, various types of optical and electrical temperature sensors. Temperature sensors herein can include contact-type temperature sensors and non-contact type temperature sensors. Optical temperature sensors herein can include infrared optical temperature sensors. Some optical temperature sensors can measure temperature at a distance such as a distance of millimeters or centimeters. Thus, even where temperature sensors are mounted along a lead 1906, temperature can be measured at a distance therefrom. Exemplary electrical temperature sensors can include, but are not limited to, thermistors, resistive temperature detectors, thermocouples, semiconductor based temperature sensors, and the like.

In some embodiments, the medical device system can include a temperature sensor disposed remotely from the medical device. A remote temperature sensor can provide temperature data in addition to or in replace of temperature sensors in other areas such as along the leads 1906. In some embodiments, a remote temperature sensor can be used to gather a core or reference temperature of the patient into which the system is implanted.

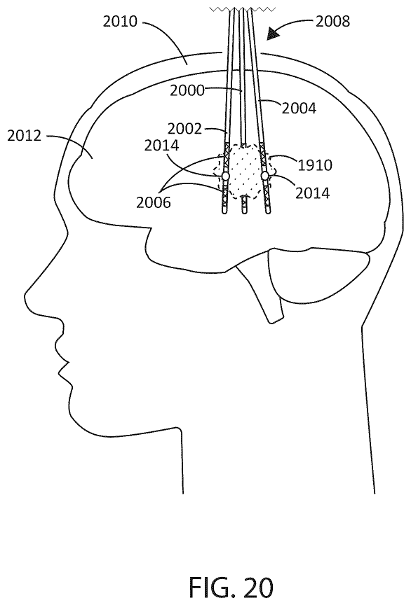

In some embodiments, the medical device can include a plurality of therapy leads implanted at or near a site a cancerous tumor or tumor resection. Referring now to FIG. 20, a schematic view of a placement of various cancer therapy leads 2000, 2002, 2004 in a region of a tumor resection site 1910 or tumor is shown in accordance with various embodiments herein. In the example of FIG. 20, temperature sensors 2014 are disposed on the therapy leads. The temperature sensors 2014 can be of any type described elsewhere herein. In the embodiment shown in FIG. 20, each cancer therapy lead includes two or more supply electrodes 2006 disposed along a length of the cancer therapy leads. Each cancer therapy lead includes a proximal and a distal supply electrode.

The side view shown in FIG. 20 also includes the placement of cancer therapy leads 2000, 2002, and 2004 around the tumor resection site 1910 and in position within a burr hole 2008 entry point on the patient's skull 2010 and into the patient's brain 2012. It will be appreciated that in some embodiments one burr hole can be used with one or more (e.g., one, two, three, or more) leads and/or electrodes. In some embodiments, multiple burr holes can be used each with one or more (e.g., one, two, three, or more) leads and/or electrodes.

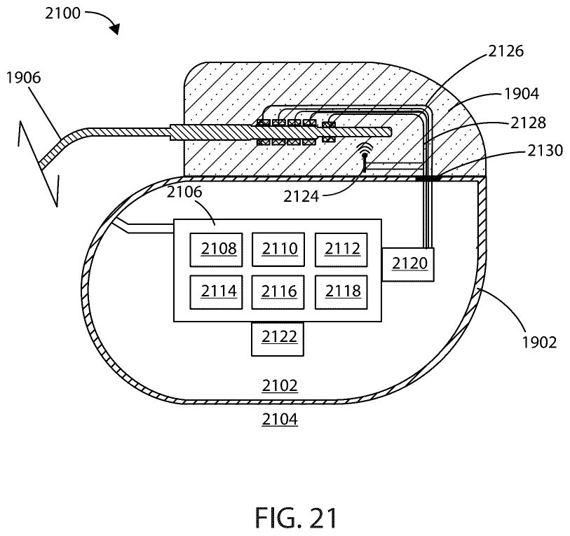

Referring now to FIG. 21, a schematic view of aspects exemplary medical device 2100 is shown in accordance with various embodiments herein. The housing 1902 can define an interior volume 2102 that can be hollow and that in some embodiments is hermetically sealed off from the area 2104 outside of medical device 2100. In other embodiments the housing 1902 can be filled with components and/or structural materials such that it is non-hollow. The medical device 2100 can include control circuitry 2106, which can include various components 2108, 2110, 2112, 2114, 2116, and 2118 disposed within housing 1902. In some embodiments, these components can be integrated and in other embodiments these components can be separate. In yet other embodiments, there can be a combination of both integrated and separate components. The medical device 2100 can also include an antenna 2124, to allow for unidirectional or bidirectional wireless data communication, such as with an external device or an external power supply. In some embodiments, the components of medical device 2100 can include an inductive energy receiver coil (not shown) communicatively coupled or attached thereto to facilitate transcutaneous recharging of the medical device via recharging circuitry.

The various components 2108, 2110, 2112, 2114, 2116, and 2118 of control circuitry 2106 can include, but are not limited to, a microprocessor, memory circuit (such as random access memory (RAM), read only memory (ROM)) and/or Electrically Erasable ROM (EEROM/Flash), recorder circuitry, controller circuit, a telemetry circuit, a power supply circuit (such as a battery), a timing circuit, and an application specific integrated circuit (ASIC), a recharging circuit, amongst others. Control circuitry 2106 can be in communication with TTF electrical field generating circuitry 2120 that can be configured to generate electric current to create one or more fields. The TTF electrical field generating circuitry 2120 can be integrated with the control circuitry 2106 or can be a separate component from control circuitry 2106. Control circuitry 2106 can be configured to control delivery of electric current from the TTF electrical field generating circuit 2120. Details of the TTF electrical field generating circuitry 2120 are described in greater detail below.

In various embodiments, one or more leads 1906 can be in electrical communication with the TTF electrical field generating circuit 2120. The one or more leads 1906 can include one or more electrodes. In some embodiments, various electrical conductors, such as electrical conductors 2126 and 2128, can pass from the header 1904 through a feed-through structure 2130 and into the interior volume 2102 of medical device 2100. As such, the electrical conductors 2126 and 2128 can serve to provide electrical connection between the one or more leads 1906 and control circuitry 2106 disposed within the interior volume 2102 of the housing 1902. In some embodiments, the control circuitry 2106 can be programmed and electronically configured to direct the TTF electrical field generating circuit 2120 to deliver an TTF electrical field via lead(s) 1706 distal electrodes and/or the housing 1902 to the site of a targeted tissue for therapy (e.g., a cancerous tumor located within a bodily tissue, a tumor resection site, or another area targeted for therapy).

In some embodiments, medical device 2100 can include a TTF electrical field sensing circuit 2122 configured to generate a signal corresponding to sensed electric fields. Electric field sensing circuit 2122 can be integrated with control circuitry 2106 or it can be separate from control circuitry 2106. Sensing electrodes can be disposed on or adjacent to the housing of the medical device, on one or more leads connected to the housing, on a separate device implanted near or in the tumor, or any combination of these locations. In some embodiments, the electric field sensing circuit 2122 can measure the electrical potential difference (voltage) between a first electrode and a second electrode, wherein the first and second electrodes are in any of the aforementioned locations. In some embodiments, the electric field sensing circuit can be configured to measure sensed electric fields and to record electric field strength in e.g., V/cm.

In some embodiments, recorder circuitry can be configured to record the data produced by the electric field sensing circuit 2122 and record time stamps regarding the same. In some embodiments, the control circuitry 2106 can be hardwired to execute various functions, while in other embodiments the control circuitry 2106 can be directed to implement instructions executing on a microprocessor or other external computation device. A wireless communication interface can also be provided for communicating with external computation devices such as a programmer, a home-based unit, and/or a mobile unit (e.g., a cellular phone, personal computer, smart phone, tablet computer, smartwatch, and the like).

It will be appreciated that low frequency current flow through therapy electrodes has the potential for causing neurostimulation. Embodiments herein can prevent low frequency current flow through therapy electrodes that could cause neurostimulation. Various embodiments herein can include a field-generating circuit with a ground that is DC referenced to a metal of the supply electrode (such as platinum) via a single reference resistor to the housing. By having a single DC connection between the medical device internal circuitry and patient contacting electrodes and device case, undesired DC or low frequency current flow, such as between patient therapy electrodes and/or the housing, will not occur. Also, in various embodiments, a high value resistor (such as 100K ohms or more) can be used to limit unintentional current if one of the DC blocking therapy capacitors were to fail (“short” or become “leaky”).

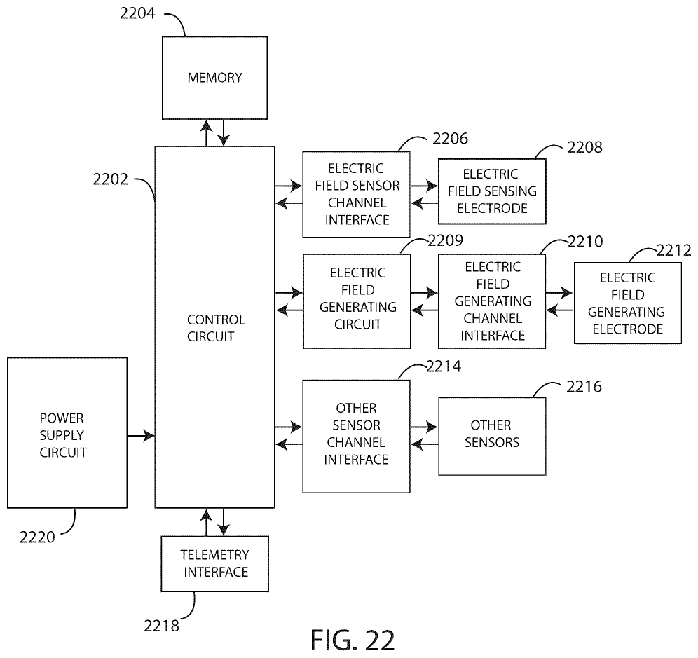

Elements of various embodiments of the medical devices described herein are shown in FIG. 22. However, it will be appreciated that some embodiments can include additional elements beyond those shown in FIG. 22. In addition, some embodiments may lack some elements shown in FIG. 22. The medical devices as embodied herein can gather information through one or more sensing channels and can output information through one or more field generating channels. A microprocessor 2202 can communicate with a memory 2204 via a bidirectional data bus. The microprocessor 2202 can be in electric communication with power supply circuit 2220. The memory 2204 can include read only memory (ROM) or random access memory (RAM) for program storage and RAM for data storage. The microprocessor 2202 can also be connected to a telemetry interface 2218 for communicating with external devices such as a programmer, a home-based unit and/or a mobile unit (e.g. a cellular phone, personal computer, smart phone, tablet computer, and the like) or directly to the cloud or another communication network as facilitated by a cellular or other data communication network. In some embodiments, the medical device can include an inductive energy receiver coil interface (not shown) communicatively coupled or attached thereto to facilitate transcutaneous recharging of the medical device.

The medical device can include one or more electric field sensing electrodes 2208 and one or more electric field sensor channel interfaces 2206 that can communicate with a port of microprocessor 2202. The medical device can also include one or more TTF electrical field generating electrodes 2212 and one or more electric field generating channel interfaces 2210 and one or more electric field generating circuits 2209 that can communicate with a port of microprocessor 2202. The medical device can also include one or more other sensors 2216, such as physiological sensors, respiration sensors, or chemical sensors, and one or more other sensor channel interfaces 2214 that can communicate with a port of microprocessor 2202. The channel interfaces 2206, 2210, and 2214 can include various components such as analog-to-digital converters for digitizing signal inputs, sensing amplifiers, registers which can be written to by the control circuitry in order to adjust the gain and threshold values for the sensing amplifiers, source drivers, modulators, demodulators, multiplexers, and the like.

In some embodiments, the physiological sensors can include sensors that monitor temperature, blood flow, blood pressure, and the like. In some embodiments, the respiration sensors can include sensors that monitor respiration rate, respiration peak amplitude, and the like. In some embodiments, the chemical sensors can measure the quantity of an analyte present in a treatment area about the sensor, including but not limited to analytes such as of blood urea nitrogen, creatinine, fibrin, fibrinogen, immunoglobulins, deoxyribonucleic acids, ribonucleic acids, potassium, sodium, chloride, calcium, magnesium, lithium, hydronium, hydrogen phosphate, bicarbonate, and the like. However, many other analytes are also contemplated herein. Exemplary chemical/analyte sensors are disclosed in commonly owned U.S. Pat. No. 7,809,441 to Kane et al., and which is hereby incorporated by reference in its entirety.

Although the other sensors 2216 are shown as part of a medical device in FIG. 22, it is realized that in some embodiments one or more of the other sensors could be physically separate from the medical device. In various embodiments, one or more of the other sensors can be within another implanted medical device communicatively coupled to a medical device via telemetry interface 2218. In yet other embodiments, one or more of the other sensors can be external to the body and coupled to a medical device via telemetry interface 2218. In some embodiments, the other sensors can include drug delivery sensors, biopsy apparatus sensors, optical sensors, or irrigation sensors.

In some embodiments, the medical devices herein can include an TTF electrical field generating circuit configured to generate one or more TTF electrical fields at or near a site of the cancerous tumor. The medical devices herein can include control circuitry in communication with the TTF electrical field generating circuit, the control circuitry configured to control delivery of the one or more TTF electrical fields from the TTF electrical field generating circuit at or near the site of the cancerous tumor. The control circuitry can cause the TTF electrical field generating circuit to generate one or more TTF electrical fields at frequencies selected from a range of between 10 kHz to 1 MHz at the site of a cancerous tumor located within a bodily tissue, the one or more TTF electrical fields effective to delay mitosis and cause mitotic synchronization within a proportion of the cancerous cell population. In some embodiments, the medical device further can include one or more electrical leads in electrical communication with the TTF electrical field generating circuit.

In some embodiments, the medical devices herein include a medical device system for treating a cancerous tumor. The medical device housing can include a TTF electrical field generating circuit configured to generate one or more TTF electrical fields at or near a site of the cancerous tumor, the cancerous tumor including a cancerous cell population. The medical device system can include control circuitry in communication with the TTF electrical field generating circuit, where the control circuitry is configured to control delivery of the one or more TTF electrical fields from the TTF electrical field generating circuit at or near the site of the cancerous tumor. The medical device system can include a drug delivery catheter for administering one or more chemotherapeutic agents at or near the site of the cancerous tumor. The control circuitry of the medical device system causes the TTF electrical field generating circuit to generate one or more TTF electrical fields at frequencies selected from a range of between 10 kHz to 1 MHz at the site of a cancerous tumor located within a bodily tissue, the one or more TTF electrical fields effective to delay mitosis and cause mitotic synchronization within a proportion of the cancerous cell population.

In some embodiments, the medical devices herein can include a medical device for treating a cancerous tumor located within a subject. The medical device can include a TTF electrical field generating circuit configured to generate one or more TTF electrical fields at or near a site of the cancerous tumor, the cancerous tumor including a cancerous cell population. The medical device can include control circuitry in communication with the TTF electrical field generating circuit. The control circuitry of the medical devices controls delivery of the one or more TTF electrical fields from the TTF electrical field generating circuit at or near the site of the cancerous tumor by following a predefined schedule that causes the TTF electrical fields to vary in at least one of intensity and frequency over the course of a defined time period of at least six hours.

The medical devices herein can include a medical device of treating a cancerous tumor, including one or more implantable electrodes configured for placement on the inside of a body of a subject with the cancerous tumor. The medical device can include one or more external electrodes configured for placement on an outside surface of the body of the subject. The medical device can include a TTF electrical field generating circuit configured for generating a TTF electrical field between at least one pair of electrodes according to a predefined schedule, the TTF electrical field having frequencies within a range of between 10 kHz to 1 MHz. The medical device can include control circuitry configured for receiving a pause command from the subject, wherein the pause command causes cessation of generating the TTF electrical field.

### Chemotherapeutic Agents

One or more chemotherapeutic agents can be suitable for use with the methods and devices described herein. therapeutic agents suitable for use herein can include, but are not limited to, antimitotic agents, alkylating agents, antimetabolites, kinase inhibitors, proteasome inhibitors, histone deacetylase inhibitors, topoisomerase inhibitors, poly(ADP-ribose) polymerase (PARP) inhibitors, antitumor antibiotics, retinoids, and combination drugs.

The anti-mitotic agents can include anti-mitotic agents that act on the microtubules (i.e., spindle fiber) present during mitosis. Suitable anti-mitotic agents can include those that have microtubule-stabilizing properties or those that have microtubule-destabilizing properties. Without wishing to be bound by any particular theories, it is believed that anti-mitotic agents including those that have microtuble-stablizing properties or those that have microtubule-destabilizing properties act on various domains of either alpha-tubulin or beta-tubulin proteins that make up the microtubule structure.

In some embodiments, the anti-mitotic agents herein can include anti-mitotic agents that act on the contractile ring, which can include, but are not to be limited to F-actin, myosin-2, anillin, one or more septins, Rho, profilin, cofilin, and male germ cell Ras-related C3 botulinum toxin substrate GTPase activating proteins (MgcRacGAP). In other embodiments, the anti-mitotic agents can include anti-miotic agents that act on nuclei acids, such as DNA and RNA. Suitable anti-mitotic agents can include those that have contractile ring-stabilizing properties or those that have contractile ring-destabilizing properties. Without wishing to be bound by any particular theories, it is believed that anti-mitotic agents including those that have contractile ring-stabilizing properties or those that have contractile ring-destabilizing properties act on various domains of the proteins that form the contractile ring, as discussed herein. Anti-mitotic agents suitable for use herein include, but are not to be limited to, at least one of vindesine (such as Eldisine and Fildesin), vincristine suflate (such as Vincasar PFS and Oncovin), vincristine liposome (such as Marqibo), vinblastine (such as Velban), paclitaxel (such as Onxol and Taxol), pacliaxel albumin-stabilized nanoparticle formulation (such as Abraxane), vinorelbine tartrate (such as Nabelbine), docetaxel (such as Taxotere and Docefrez), cribulin mesylate (such as Halaven), cabazitaxel (such as Jevtana), ixabepilone (such as Ixempra), vinflunine (such as Javlor), 2-methoxyestradiol, patupilone, and derivatives thereof. In some embodiments, the chemotherapeutic agents herein include those that have a therapeutic half-life of less than 24 hours. In some embodiments, the chemotherapeutic agents herein include those that have a therapeutic half-life of less than 48 hours. In some embodiments, the chemotherapeutic agents herein include those that have a therapeutic half-life of less than 60 hours.

In some embodiments, alkylating agents can be suitable for use herein. Exemplary alkylating agents can include nitrogen mustards, alkyl sulfonates, nitrosourceas, triazines, platinum, and others. Suitable nitrogen mustards can include, but are not limited to, at least one of chlorambucil (such as Leukeran), bendamustine hydrochloride (such as Treanda, Bendeka, Vivimusta, and Belrapzo), ifosfamide (such as Ifex), cyclophosphamide (such as Cytoxan), melphan hydrochloride (such as Evomela), melphan (such as Alkeran), mechlorethamine hydrochloride, also known as chlormethine (such as Mustargen and Valchlor), cyclophosphamide (such as Neosar and Cytoxan Lyophilized), melphan Flufenamide (such as Pepaxto and Pepaxti), trofosfamide (such as Ixoten), estramustine (such as Emcyt and Estracyt), and derivatives thereof. Exemplary alkyl sulfonates can include, but are not limited to, at least one of busulfan (such as Myleran and Busulfex, treosulfan (such as Trecondi), and derivatives thereof. Exemplary nitrosourceas can include, but are not limited to, at least one of carmustine (such as Gliadel and BiCNU), lomustine (such as Gleostine and CeeBU), streptozocin (such as Zanosar), fotemustine (such as Mustophoran), ranimustine (such as Cymerin), and derivatives thereof. Exemplary triazines can include, but are not limited to, at least one of temozolomide (such as Temodar), dacarbazine (such as DTIC-Dome), altretamine (such as Hexalen), procarbazine hydrochloride (such as Matulane), and derivatives thereof. Exemplary platinum agents can include, but are not limited to, at least one of carboplatin (such as Paraplatin), cisplatin (such as Platinol and Platinol-AQ), oxaliplatin (such as Eloxatin), and derivatives thereof.

The platinum chemotherapeutic agents herein can include optically activated chemotherapeutic agent. The optically activated chemotherapeutic agents can include, but are not to be limited to, photoactivated platinum compounds or photoactivated photostatin compounds. In some embodiments, the photoactivated photostatin compounds include light-activated combretastatin A-4, and analogs and derivatives thereof. In some embodiments, the optically activated chemotherapeutic agents can be optically activated by visible light emitted by optical emitters present on various leads described herein within the range of 350 nm to 850 nm. In some embodiments, the optically activated chemotherapeutic agents can be optically activated by visible light within the range of 450 nm to 650 nm. The optically activated chemotherapeutic agents herein can include those that are optically activated by a wavelength that can be greater than or equal to 350 nm, 400 nm, 450 nm, 500 nm, 550 nm, 600 nm, 650 nm, 700 nm, 750 nm, 800 nm, or 850 nm, or can be an amount falling in a range within any of the foregoing. In some embodiments, the optically activated chemotherapeutic agents herein can be optically inactivated by visible light within the range of 350 nm to 850 nm. In some embodiments, the if the optically activated chemotherapeutic agents herein are optically inactivated by visible light within the range of 350 nm to 850 nm, the can be further activated by visible light within the range of 350 nm to 850 nm for multiple cycles.

Suitable optically activated chemotherapeutic agents include, but are not to be limited to, photoactivated platinum compounds or photoactivated photostatin compounds. In some embodiments, the photoactivated photostatin compounds include light-activated combretastatin A-4, and analogs and derivatives thereof.

It is herein contemplated that other alkylating agents can be suitable for use with the methods and devices described herein. Exemplary alkylating agents can include, but are not limited to, at least one of lurbinectedin (such as Zepzelca), trabectedin (such as Yondelis), pipobroman (such as Vercite and Vercyte), thiotepa (such as Thioplex and Tepadina), and deratives thereof.

Antimetabolites suitable for use herein, can include, but are not limited to, at least one of capecitabine (such as Xeloda), hydroxyurea, also known as hydroxycarbamide (such as Hydrea, Siklos, Mylocel, and Droxia), gemcitabine hydrochloride (such as Gemzar and Infugem), methotrexate sodium (such as Trexall, Xatmep, Otrexup, Rheumatrex dose pack, and Rasuvo), mercaptopurine (such as Purinethol, Purixan, and Xaluprine), cladribine (such as Mavenclad and Leustatin), pralatrexate (such as Folotyn), fludarabine (such as Fludara and Oforta), pemetrexed (such as Alimta) (such as Pemfexy), phioguanine, also known as tioguanine (such as Tabloid), floxuridine (such as FUDR), cytarabine liposomal (such as DepoCyt), decitabine (such as Dacogen), clofarabine (such as Clolar), nelarabine (such as Arranon), fluorouracil (such as Adrucil, Tolak, Carac, Efudex, 5-FU, Fluoroplex), azacitidine (such as Onureg and Vidaza), azathioprine, carmofur, pentostatin (such as Nipent), raltitrexed (such as Tomudex), cytarabine arabinoside (such as ara-C), and derivatives thereof.

Kinase inhibitors c tyrosine kinase inhibitors and serine-threonine kinase inhibitors. Exemplary tyrosine kinase inhibitors can include, but are not limited to, at least one of imatinib mesylate (such as Gleevec), dasatinib (such as Sprycel), nilotinib (such as Tasigna), bosutinib (such as Bosulif), ponatinib hydrochloride (such as Iclusig), asciminib hydrochloride (such as Scemblix), gefitinib (such as Iressa), erlotinib hydrochloride (such as Tarceva), sunitinib malate (such as Sutent), sorafenib tosylate (such as Nexavar), lapatinib ditosylate (such as Tykerb), pazopanib hydrochloride (such as Votrient), regorafenib (such as Stivarga), vandetanib (such as Caprelsa), crizotinib (such as Xalkori), cabozantinib-S-malate (such as Cometriq and Cabometyx), axitinib (such as Inlyta), acalabtrutinib (such as Calquence), afatinib dimaleate (such as Gilotrif), alectinib (such as Alecensa), brigatinib (such as Alunbrig), avapritinib (such as Ayvakit), zanubrutinib (such as Brukinsa), capmatinib hydrochlrodei (such as Tabrecta), ceritinib (such as Zykadia), dacomitinib (such as Vizimpro), entrecetinib (such as Rozlytrek), mobocertinib succinate (such as Exkivity), fedratinib hydrochloride (such as Inrebic), tivozanib hydrochloride (such as Fotivda), futibatinib (such as Lytgobi), pralsetinib (such as Gavreto), gilteritinib fumarate (such as Xospata), ibrutinib (such as Imbruvica), infigratinib phosphate (such as Truseltiq), ruxolitinib phosphate (such as Jakafi), pirtobrutinib (such as Jaypirca), lorlatinib (such as Lorbrena), erdafitinib (such as Balversa), midostaurin (such as Rydapt), neratinib maleate (such as Nerlynx), osimertinib mesylate (such as Tagrisso), pacritinib citrate (such as Vonjo), pemigatinib (such as Pemazyre), ripretinib (such as Qinlock), selpercatinib (such as Retevmo), tepotinib Hydrochloride (such as Tepmetko), tucatinib (such as Tukysa), nintedanib (such as Ofev and Vargatef), lenvatinib mesylate (such as Lenvima), ziv-aflibercept (such as Zaltrap), and derivatives thereof.

Exemplary serine/threonine kinase inhibitors can include, but are not limited to, at least one of vemurafenib (such as Zelboraf), abemaciclib (such as Verzenio), encorafenib (such as Braftovi), dabrafenib mesylate (such as Tafinlar), palbociclib (such as Ibrance), ribociclib (such as Kisqali), larotrectinib sulfate (such as Vitrakvi), everolimus (such as Afinitor), and derivatives thereof.

It is contemplated herein that other kinase inhibitors can be suitable for the methods and devices described herein. Exemplary kinase inhibitors can include, but are not limited to, at least one of vismodegib (such as Erivedge), adagrasib (such as Krazati), copanlisib hydrochloride (such as Aliqopa), alpelisib (such as Piqray), binimetinib (such as Mektovi), cobimetinib fumarate (such as Cotellic), duvelisib (such as Copiktra), idelalisib (such as Zydelig), selumetinib sulfate (such as Koselugo), trametinib dimethyl sulfoxide (such as Mekinist), umbralisib (such as Ukoniq), temsirolimus (such as Torisel), sirolimus protein-bound particles (such as Fyarro), and derivatives thereof.

Proteasome inhibitors suitable for use herein can include, but are not limited to, at least one of ixazomib citrate (such as Ninlaro), carfilzomib (such as Kyprolis), bortezomib (such as Velcade), and derivatives thereof.

Histone deacetylase inhibitors suitable for use herein can include, but are not limited to, at least one of vorinostat (such as Zolinza), romidepsin (such as Istodax), belinostat (such as Belcodaq), panobinostat (such as Farydak), and derivatives thereof.

Topoisomerase inhibitors suitable for use herein can include camptothecin analogs and podophyllotoxin derivatives. Exemplary camptothecin analogs can include, but are not limited to, at least one of irinotecan hydrochloride (such as Camptosar), irinotecan hydrochloride liposome (such as Onivyde), topotecan hydrochloride (such as Hycamtin), belotecan (such as Camptobell), and derivatives thereof. Exemplary pdophyllotoxin derivatives can include, but are not limited to, at least one of etoposide (such as VePesid) (such as Toposar), etoposide phosphate (such as Etopophos), teniposide (such as Vumon), and derivatives thereof.

It is contemplated herein that other topoisomerase inhibitors can be suitable for user herein. Exemplary topoisomerase inhibitors can include, but are not limited to, at least one of fam-Trastuzumab Deruxtecan-nxki (such as Enhertu), mitoxantrone (such as Novantrone), telotristat etiprate (such as Xermelo), and derivatives thereof.

PARP inhibitors suitable for use herein can include, but are not limited to, at least one of rucaparib camsylate (such as Rubraca), olaparib (such as Lynparza), niraparib tosylate monohydrate (such as Zejula), talazorparib tosylate (such as Talzenna), and derivatives thereof.

Antitumor antibiotics suitable for use herein can include anthracyclines (such as topoisomerase II inhibitors) and others. Exemplary anthracyclines can include, but are not limited to, at least one of doxorubicin hydrochloride (such as Adriamycin), doxorubicin hydrochloride liposome (such as Doxil), epirubicin (such as Ellence), daunorubicin hydrochloride (such as Cerubidine and Rubidomycin), idarubicin hydrochloride (such as Idamycin PFS), valrubicin (such as Valstar), aclarubicin (such as Aclacin, Aclacinomycine, Aclacinon, Aclasplastin, and Jaclacin), amrubicin (such as Calsed), pirarubicin, and derivatives thereof. Other exemplary antitumor antibiotics can include, but are not limited to, at least one of bleomycin sulfate (such as Blenoxane), dactinomycin (such as Cosmegen), mitomycin (such as Jelmyto), plicamycin (such as Mithracin), pixantrone (such as Pixuvri), amsacrine (such as Amsidine), and derivatives thereof.

Retinoids suitable for use herein can include, but are not limited to, at least one of bexarotene (such as Targretin), alitretinoin (such as Panretin), tretinoin (such as Vesamoid), acitretin (such as Soriatane), and derivatives thereof.

It is contemplated herein that other chemotherapeutic agents can be suitable for the methods and devices described herein. Other chemotherapeutic agents can include, but are not limited to, at least one of arsenic trioxide (such as Trisenox), asparaginase Erwinia chrysanthemi (such as Rylaze), calasparagase pegol-mknl (such as Asparalas), mitotane (such as Lysodren), omacetaxine mepesuccinate (such as Ceflatonin and Synribo), pegaspargase (such as Oncaspar), belzutifan (such as Welireg), aminolevulinic acid hydrochloride (such as Ameluz and Levulan Kerastick), pamidronate disodium (such as Aredia), iobenguane 131 (such as Azedra), glasdegib maleate (such as Daurismo), denilcukin diftitox (such as Ontak), tagraxofusp-erzs (such as Elzonis), enasidenib mesylate (such as Idhifa), propranolol hydrochloride (such as Hemangeol and Inderal), ivosidenib (such as Tibsovo), sotorasib (such as Lumakras), lutetium Lu 177-Dotatate (such as Lutathera), lutetium Lu 177 Vipivotide Tetraxetan (such as Pluvicto), lutetium LU 177-edotreotide, sonidegib (such as Odomzo), radium 223 dichloride (such as Xofigo), qlutasidenib (such as Rezlidhia), selinexor (such as Xpovio), tazemetostat hydrobromide (such as Tazverik), ventoclax (such as Venclexta), zoledronic acid (such as Reclast and Zometa), realgar/indigo naturalis formulation, venetoclax (such as Venclexta and Venclyxto), methylene blue, also known as methylthioninium chloride, and derivatives thereof.

It is further contemplated herein that combinations of chemotherapeutic agents can be suitable for use herein. Exemplary combinations can include, but are not limited to, at least one of ABVD (doxorubicin hydrochloride, bleomycin, vinblastine sulfate, dacarbazine), ABVE (doxorubicin hydrochloride, bleomycin, vincristine sulfate, etoposide phosphate), ABVE-PC (doxorubicin hydrochloride, bleomycin, vincristine sulfate, etoposide phosphate, prednisone, cyclophosphamide), AC (doxorubicin hydrochloride, cyclophosphamide), AC-T (doxorubicin hydrochloride, cyclophosphamide, paclitaxel), ADE (Cytarabine, daunorubicin hydrochloride, ctoposide phosphate), BEACOPP (bleomycin, ctoposide phosphate, doxorubicin hydrochloride, cyclophosphamide, vincristine sulfate, procarbazine hydrochloride, prednisone), BEP (bleomycin, etoposide phosphate, cisplatin), BuMel (busulfan, melphalan hydrochloride), CAF (Cyclophosphamide, doxorubicin hydrochloride, fluorouracil), CAPOX (capecitabine, oxaliplatin), Carboplatin-taxol (carboplatin, paclitaxel), CEM (carboplatin, ctoposide phosphate, melphalan hydrochloride), CEV (carboplatin, ctoposide phosphate, vincristine sulfate), Chlorambucil-prednisone (chlorambucil, prednisone), CHOP (cyclophosphamide, doxorubicin hydrochloride, vincristine sulfate, prednisone), CMF (cyclophosphamide, methotrexate, fluorouracil), COPDAC (cyclophosphamide, vincristine sulfate, prednisone, dacarbazine), COPP (cyclophosphamide, vincristine sulfate, procarbazine hydrochloride, prednisone), COPP-ABV (cyclophosphamide, vincristine sulfate, procarbazine hydrochloride, prednisone, doxorubicin hydrochloride, bleomycin, vinblastine sulfate), CVP (cyclophosphamide, vincristine sulfate, prednisone), daunorubicin hydrochloride and cytarabine liposome (such as Vyxcos), decitabine and cedazuridine (such as Inqovi), EPOCH (etoposide phosphate, prednisone, vincristine sulfate, cyclophosphamide, doxorubicin hydrochloride), FEC (fluorouracil, epirubicin hydrochloride, cyclophosphamide), FOLFIRI (leucovorin calcium, fluorouracil, irinotecan hydrochloride), FOLFIRI-Bevacizumab (leucovorin calcium, fluorouracil, irinotecan hydrochloride, bevacizumab), FOLFIRI-Cetuximab (leucovorin calcium, fluorouracil, irinotecan hydrochloride, cetuximab), FOLFIRINOX (leucovorin calcium, fluorouracil, irinotecan hydrochloride, oxaliplatin), FOLFOX (leucovorin calcium, fluorouracil, oxaliplatin), FU-LV (fluorouracil, leucovorin calcium), Gemcitabine-cisplatin (gemcitabine hydrochloride, cisplatin), Gemcitabine-oxaliplatin (gemcitabine hydrochloride, oxaliplatin), hyper-CVAD (cyclophosphamide, vincristine sulfate, doxorubicin hydrochloride, dexamethasone), ICE (ifosfamide, carboplatin, ctoposide phosphate), JEB (carboplatin, etoposide phosphate, bleomycin), trifluridine and tipiracil hydrochloride (such as Lonsurf), MOPP (mechlorethamine hydrochloride, vincristine sulfate, procarbazine hydrochloride, prednisone), MVAC (methotrexate, vinblastine sulfate, doxorubicin hydrochloride, cisplatin), OEPA (vincristine sulfate, etoposide phosphate, prednisone, doxorubicin hydrochloride), OFF (oxaliplatin, fluorouracil, leucovorin calcium), OPPA (vincristine sulfate, procarbazine hydrochloride, prednisone, doxorubicin hydrochloride), PAD (bortezomib, doxorubicin hydrochloride, dexamethasone), PCV (procarbazine hydrochloride, lomustine, vincristine sulfate), PEB (cisplatin, etoposide phosphate, bleomycin), R-CHOP (rituximab, cyclophosphamide, doxorubicin hydrochloride, vincristine sulfate, prednisone), R-CVP (rituximab, cyclophosphamide, vincristine sulfate, prednisone), R-EPOCH (rituximab, etoposide phosphate, prednisone, vincristine sulfate, cyclophosphamide, doxorubicin hydrochloride), R-ICE (rituximab, ifosfamide, carboplatin, ctoposide phosphate), STANDORD V (mechlorethamine hydrochloride, doxorubicin hydrochloride, vinblastine sulfate, vincristine sulfate, bleomycin, etoposide phosphate, prednisone), TAC (docetaxel, doxorubicin hydrochloride, cyclophosphamide), TPF (docetaxel, cisplatin, fluorouracil), VAC (vincristine sulfate, dactinomycin, cyclophosphamide), VAMP (vincristine sulfate, doxorubicin hydrochloride, methotrexate, prednisone), VelP (vinblastine sulfate, ifosfamide, cisplatin), VIP (etoposide, ifosfamide, cisplatin), XELIRI (capecitabine, irinotecan hydrochloride), XELOX (capecitabine, oxaliplatin), tegafur, gimeracil, oteracil (such as Teysuno).

The chemotherapeutic agents herein can be administered at or near the site of the cancerous tumor in a therapeutically effective dose. In some embodiments, the chemotherapeutic agents herein can be administered away from the site of the cancerous tumor into the systemic circulation in a therapeutically effective dose. The chemotherapeutic agents can be administered through a transcutaneous access port that is in fluid communication with the systemic venous system anywhere on the subject's body. In other embodiments, the chemotherapeutic agents can be administered orally in the form of a pill or capsule. In other embodiments, the chemotherapeutic agents can be topically administered to a patient's skin in the form of a cream or ointment. In other embodiments, the chemotherapeutic agents can be administered via injection into a patient's muscle, skin, or into the cancerous tumor. In some embodiments, the chemotherapeutic agents can be administered directly into a patient's artery that supplies blood to the cancerous tumor. In other embodiments, the chemotherapeutic agents can be administered as a polymer wafer implanted into a patient. In other embodiments, the chemotherapeutic agents can be administered into a patient's cerebrospinal fluid (CSF). In other embodiments, the chemotherapeutic agents can be administered using magnetic cationic microspheres loaded with chemotherapeutic agents delivered into a patient via a catheter at or near a site of a cancerous tumor.

In some embodiments, it is believed hyperbaric oxygen therapy (HBOT) can enhance the effectiveness of chemotherapeutic drugs by increasing oxygenation in the cancerous tumor tissue. HBOT can include placing a patient in a pressurized chamber for a desired length of time and breathing 100% oxygen.

The methods herein can include a method for treating a cancerous tumor located within a subject including applying one or more TTF electrical fields at or near a site of the cancerous tumor, the cancerous tumor including a cancerous cell population. The method can include removing the one or more TTF electrical fields and administering an optically activated chemotherapeutic agent at or near a site of the cancerous tumor after the one or more TTF electrical fields have been removed. The method can include irradiating the optically activated chemotherapeutic agent by delivering photoactivating light energy at or near the site of the cancerous tumor. In some embodiments, the optically activated chemotherapeutic agent is administered at a therapeutically effective dose of optically activated chemotherapeutic agent for release at or near the site of the cancerous tumor.

The medical devices herein can include treating a cancerous tumor located within a subject including a TTF electrical field generating circuit configured to generate one or more TTF electrical fields at or near a site of the cancerous tumor, the cancerous tumor including a cancerous cell population. The medical device can include control circuitry in communication with the TTF electrical field generating circuit, the control circuitry configured to control delivery of the one or more TTF electrical fields from the TTF electrical field generating circuit at or near the site of the cancerous tumor. The medical device can include a catheter configured to administer an optically activated chemotherapeutic agent at or near a site of the cancerous tumor. The medical device can include one or more optical emitters configured to irradiate the optically activated chemotherapeutic agent by delivering photoactivating light energy at or near the site of the cancerous tumor.

The chemotherapeutic agents herein can include nanoparticles. In some embodiments, the nanoparticles are made from a polymer, such as a biodegradable polymer. In some embodiments, the nanoparticles can include an effective amount of chemotherapeutic agent for release at or near the site of the cancerous tumor. In some embodiments, the nanoparticles include at least one of the vindesine, vincristine, vinblastine, paclitaxel, docetaxel, 2-methoxyestradiol, patupilone, trastuzumab emtansine, and derivatives thereof. The nanoparticles can be released at or near the site of a cancerous tumor to deliver a therapeutically effective dose of optically active chemotherapeutic agent. In some embodiments, the nanoparticles can include an effective amount of optically activated chemotherapeutic agent, as described elsewhere herein.

The methods herein can include a method for treating a cancerous tumor located within a subject, including applying one or more TTF electrical fields at or near a site of the cancerous tumor, the cancerous tumor including a cancerous cell population. The method can include administering nanoparticles including a chemotherapeutic agent at or near the site of the cancerous tumor. In some embodiments, the method can include removing the one or more TTF electrical fields before administering the nanoparticles at or near the site of the cancerous tumor.

The medical devices herein can include a medical device for treating a cancerous tumor located within a subject including a TTF electrical field generating circuit configured to generate one or more TTF electrical fields at or near a site of the cancerous tumor, the cancerous tumor including a cancerous cell population. The medical device can include control circuitry in communication with the TTF electrical field generating circuit, the control circuitry configured to control delivery of the one or more TTF electrical fields from the TTF electrical field generating circuit at or near the site of the cancerous tumor. The medical device can include a catheter configured to administer an nanoparticles at or near a site of the cancerous tumor.

### Hormone/Biologic Therapeutic Agents

One or more additional agents can be suitable for use with the methods and devices described herein. It is contemplated herein, hormone/biologic therapeutic agents can be administered simultaneously to a patient receiving electrical therapy, before the patient receives electrical therapy, and/or after the patient receives electrical therapy. It is further contemplated herein, hormone/biologic therapeutic agents can be administered to a patient simultaneously to other treatment modalities such as photodynamic therapy, stem cell transplant therapy, cryotherapy, immunotherapy, chemotherapy, radiation therapy, gene therapy, telomerase inhibition therapy, and/or surgery. In other embodiments, hormone/biologic therapeutic agents can be administered to a patient before or after the other treatment modalities listed above.

By way of example, a hormone/biologic therapeutic agent can be combined with an alternating TTF electrical field therapy. Suitable hormone/biologic therapeutic agents can include, but are not to be limited to, progestins, steroidal antiestrogens, luteinizing hormone-releasing hormone agonists, gonadotropin-releasing hormone agonists (also known as luteinizing hormone-releasing hormone antagonists), antiandrogens, aromatase inhibitors, selective estrogen receptor modulators, corticosteroids, somatostatin analogues, prolactin lowering agents, thyrotropin stimulating hormone agonist, and others. In some embodiments, progestins can include one or more agents that are synthetic forms of the hormone progesterone used to increase progesterone levels in a patient. In some embodiments, steroidal antiestrogens can include one or more agents that prevent the estrogen hormone from binding to estrogen receptors, thereby decreasing estrogen levels in a patient. In some embodiments, luteinizing hormone-releasing hormone agonists can include one or more agents that are synthetic analogs of luteinizing hormone-releasing hormone used to decrease testosterone levels in a patient. In some embodiments, gonadotropin-releasing hormone agonists can include one or more agents that are synthetic analogs of gonadotropin-releasing hormone used to decrease testosterone levels in a patient. In some embodiments, antiandrogens can include one or more agents that block the effects of androgens on cancerous cells. In some embodiments, aromatase inhibitors can include one or more agents that block the enzyme aromatase to decrease estrogen levels in a patient. In some embodiments, selective estrogen receptor modulators can include one or more agents that block the effect of estrogen on cancerous cells. In some embodiments, corticosteroids can include one or more agents that are natural hormone and hormone-like agents that can suppress a patient's immune system. In some embodiments, somatostatin analogues can include one or more agents that are somatostatin analogues used to inhibit growth hormones such as glucagon and insulin. In some embodiments, prolactin lowering agents can include one or more agents that decrease the levels of prolactin in a patient. In some embodiments, thyrotropin stimulating hormone agonist can include one or more agents that stimulates the thyroid gland and increases the metabolism of tissues in the patient.

Exemplary progestins can include, but are not limited to, at least one of megestrol acetate and medroxyprogesterone. Exemplary steroidal antiestrogens can include, but are not limited to, ulvestrant (such as Faslodex), and derivatives thereof. Exemplary luteinizing hormone-releasing hormone agonists can include, but are not limited to, at least one of, leuprolide acetate (such as Eligard) (such as Lupron Depot), goserelin acetate (such as Zoladex), buserelin (such as Suprefact), and derivatives thereof. Exemplary gonadotropin-releasing hormone agonists can include, but are not limited to, at least one of histrelin (such as Vantas) (such as Supprelin LA), triptorelin (such as Trelstar), linzagolix (such as Yselty), degarelix (such as Firmagon), and derivatives thereof. Exemplary antiandrogens can include, but are not limited to, at least one of abiraterone acetate (such as Zytiga) (such as Yonsa), apalutamide (such as Erleada), bicalutamide (such as Casodex), enzalutamide (such as Xtandi), flutamide (such as Eulexin), nilutamide (such as Nilandron), darolutamide (such as Nubeqa), and derivatives thereof. Exemplary aromatase inhibitors can include, but are not limited to, at least one of anastrozole (such as Arimidex), exemestane (such as Aromasin), letrozole (such as Femara), and derivatives thereof. Exemplary selective estrogen receptor modulators can include, but are not limited to, at least one of tamoxifen citrate (such as Soltamox), raloxifene hydrochloride (such as Evista), toremifene (such as Fareston), elacestrant dihydrochloride (such as Orserdu), and derivatives thereof. Exemplary corticosteroids can include, but are not limited to, at least one of dexamethasone (such as Decadron, Ozurdex, and Dexycu), prednisone (such as Deltasone, Orasone, and Liquid Pred), and derivatives thereof. Exemplary somatostatin analogues can include, but are not limited to, at least one of lanreotide acetate (such as Somatuline depot), octreotide (such as Sandostatin), and derivatives thereof. Exemplary prolactin lowering agents can include, but are not limited to, at least one of bromocriptine (such as Parlodel), cabergoline (such as Dostinex), quinagolide (such as Norprolac), and derivatives thereof. Exemplary thyrotropin stimulating hormone agonists can include, but are not limited to, hyrotropin alfa (such as Thyrogen), and derivatives thereof.

Other hormone or biologic agents are contemplated herein. Exemplary hormone or biologic agents can include, but are not limited to, at least one of elugolix (such as Orgovyx), fluoxymesterone (such as Androxy, Halotestin, and Ultandren), diethylstilbestrol (also known as stilbestrol), polyestradiol phosphate (such as Estradurin), ulipristal acetate (such as Ella), and derivatives thereof.

The hormone or biologic agents herein can be administered at or near the site of the cancerous tumor in a therapeutically effective dose. In other embodiments, the hormone or biologic agents can be administered away from the site of the cancerous tumor into the systemic circulation in a therapeutically effective dose. In some embodiments, the hormone or biologic agents can be administered via injection or infusion into a vein or muscle of the patient to deliver the agents directly into his/her bloodstream. In other embodiments, the hormone or biologic agents can be administered orally in the form of a pill or capsule. In other embodiments, the hormone or biologic agents can be administered through implanted devices inserted under a patient's skin, such that the agents are released into the patient's body over a certain period of time. In other embodiments, the hormone or biologic agents can be topically administered to a patient's skin in the form of a cream, gel, or patch.

### Immunologic Agents

One or more additional agents can be suitable for use with the methods and devices described herein. It is contemplated herein, immunologic agents can be administered simultaneously to a patient receiving electrical therapy, before the patient receives electrical therapy, and/or after the patient receives electrical therapy. It is further contemplated herein, immunologic agents can be administered to a patient simultaneously to other treatment modalities such as photodynamic therapy, stem cell transplant therapy, cryotherapy, hormone or biologics therapy, chemotherapy, radiation therapy, gene therapy, telomerase inhibition therapy, and/or surgery. For example, in some embodiments, one or more immunologic agents can be delivered simultaneously to the administration of an electrical therapy. Without being bound to a theory, it is believed the immunologic agents can prime the immune system and increase the treatment effectiveness. In other embodiments, immunologic agents can be administered to a patient before or after the other treatment modalities described herein.

By way of example, an immunologic agent can be combined with an alternating TTF electrical field therapy. Suitable immunologic agents can include, but are not to be limited to, monoclonal antibodies, adoptive cell therapeutic agents, cancer vaccines, immunomodulatory agents, cytokines, and others. In some embodiments, monoclonal antibodies can include one or more agents that target specific proteins found on the surface of cancer cells. In some embodiments, adoptive cell therapeutic agents can include one or more agents created using a patient's modified T-cells to express chimeric antigen receptors (CAR) that can recognize and attack cancer cells. In some embodiments, cancer vaccines can include vaccines designed to stimulate a patient's immune system to recognize and attack cancer cells. In some embodiments, immunomodulatory agents can include one or more agents that stimulate a patient's immune system. In some embodiments, cytokines can include one or more protein agents produced by immune cells that can stimulate a patient's immune system. In some embodiments, a combination of agents can be used. In other embodiments, ne or more agents can be used in conjunction with a chemotherapeutic agent during a given therapy.

Exemplary monoclonal antibodies can include checkpoint inhibitors and cluster of differentiation antigens. Exemplary checkpoint inhibitors can include, but are not limited to, at least one of programmed death-ligand 1 (PD-L1) inhibitors, cytotoxic T-lymphocyte-associated protein 4 (CTLA-4) inhibitors, programmed cell death protein 1 (PD-1) inhibitors, and lymphocyte-activation gene 3 (LAG-3) inhibitors. Exemplary PD-L1 inhibitors can include, but are not limited to, at least one of atezolizumab (such as Tecentriq), avelumab (such as Bacencio), durvalumab (such as Imfinzi), dostarlimab-gxly (such as Jemperli), and derivatives thereof. Exemplary CTLA-4 inhibitors can include, but are not limited to, at least one of ipilimumab (such as Yervoy), remelimumab-actl (such as Imjuno), and derivatives thereof. Exemplary PD-1 inhibitors can include, but are not limited to, at least one of nivolumab (such as Opdivo), pembrolizumab (such as Keytruda), pemiplimab-rwlc (such as Libtayo), tislelizumab, retifanlimab-dlwr (such as Zynyz), and derivatives thereof. Exemplary LAG-3 inhibitors can include, but are not limited to, at least one of relatlimab-rmbw, nivolumab (such as Opdualag), and derivatives thereof.

Exemplary cluster of differentiation antigens can include endoglin antigen (CD105), immunoglobulin-associated beta antigen (CD79b), campath-1 antigen (CD52), cyclic ADP ribose hydrolase (CD38), sialic acid binding lg-like lectin 3 antigen (CD33), TNF receptor superfamily member 8 (CD30), sialic acid-binding immunoglobin-type lectin 2 antigen (CD22), B-lymphocyte antigen (CD20), B-lymphocyte antigen (CD19), and cluster of differentiation 3 (CD3). Exemplary CD105 antigens can include, but are not limited to, carotuximab. Exemplary CD79b antigens can include, but are not limited to polatuzumab vedotin-piiq (such as Polivy) and derivatives thereof. Exemplary CD52 antigens can include, but are not limited to, alemtuzumab (such as Campath) and derivatives thereof. Exemplary CD38 antigens can include, but are not limited to, at least one of isatuximab-irfc (such as Sarclisa), daratumumab (such as Darzalex), and derivatives thereof. Exemplary CD33 antigens can include, but are not limited to, gemtuzumab ozogamicin (such as Mylotarg) and derivatives thereof. Exemplary CD30 antigens can include, but are not limited to, brentuximab vedotin (such as Adcetris) and derivatives thereof. Exemplary CD22 antigens can include, but are not limited to, at least one of inotuzumab ozogamicin (such as Besponsa), mosunetuzumab-axgb (such as Lunsumio), and derivatives thereof. Exemplary CD20 antigens can include, but are not limited to, at least one of rituximab (such as Riabni, Rituxan, Ruxience, and Truxima), obinutuzmab (such as Gazyva), ofatumumab (such as Arzerra), mosunetuzumab-axgb (such as Lunsumio), tositumomab, and derivatives thereof. Exemplary CD19 antigens can include, but are not limited to, at least one of blinatumomab (such as Blincyto), loncastuximab tesirine-lpyl (such as Zynlonta), and derivatives thereof. Exemplary CD3 antigens can include, but are not limited to, at least one of blinatumomab (such as Blincyto), mosunetuzumab-axgb (such as Lunsumio), teclistamab-cqyv (such as Tecvayli), catumaxomab (such as Removab), and derivatives thereof.

Other monoclonal antibodies are contemplated herein. Exemplary monoclonal antibodies can include, but are not limited to, at least one of amivantamab-vmjw (such as Rybrevant), pertuzumab (such as Perjeta), bevacizumab (such as Alymsys, Avastin, Mvasi, and Zirabev), belantamab mafodotin (such as Blenrep), mogamulizumab-kpkc (such as Poteligeo), enfortumab vedotin-ejfv (such as Padcev), margetuximab-cmkb (such as Margenza), trastuzumab (such as Herceptin), ado-Trastuzumab emtansine (such as Kadcyla), siltuximab (such as Sylvant), panitumumab (such as Vectibix), emapalumab-lzsg (such as Gamifant), naxitamab-gqgk (such as Danyelza), cetuximab (such as Erbitux), elotuzumab (such as Empliciti), dinutuximab (such as Unituxin), ibritumomab tiuxetan (such as Zevalin), necitumumab (such as Portrazza), denosumab (such as Prolia) (such as Xgeva), mirvetuximab soravtansine-gynx (such as Elahere), ravulizumab-cwvz (such as Ultomiris), tisotumab vedotin-tftv (such as Tivdak), tafasitamab-cxix (such as Monjuvi), tocilizumab (such as Actemra), ramucirumab (such as Cyramza), caplacizumab-hydp (such as Cablivi), sacituzumab govitecan-hziy (such as Trodelvy), olaratumab (such as Lartruvo), racotumomab (such as Vaxira), cculizumab (such as Soliris), belimumab (such as Benlysta), and derivatives thereof.

Exemplary adoptive cell therapeutic agents can include, but are not limited to, at least one of idecabtagene vicleucel (such as Adecma), nadofaragene firadenovec-vncg (such as Adstiladrin), lisocabtagene maraleucel (such as Breyanzi), ciltacabtagene autoleucel (such as Carvykti), talimogene laherparepvec (such as Imlygic), tisagenlecleucel (such as Kymriah), brexucabtagene autoleucel (such as Tecartus), axicabtagene ciloleucel (such as Yescarta), and derivatives thereof.

Exemplary cancer vaccines can include, but are not limited to, at least one of sipuleucel-T (such as Provenge), recombinant Human papillomarvirus bivalent vaccine (such as Cervarix), recombinant Human papillomarvirus nonvalent vaccine (such as Gardasil 9), recombinant Human papillomarvirus quadrivalent vaccine (such as Gardasil), bacillus Calmette-Guérin (BCG) vaccine, DCVax-L, ICT-107, SurVaxM, AV-GBM-1, HSPPC-96, and derivatives thereof.

Exemplary immunomodulatory agents can include, but are not limited to, at least one of thalidomide (such as Thalomid and Synovir), lenalidomide (such as Revlimid), pomalidomide (such as Pomalyst), imiquimod (such as Zyclara) (such as Aldara), pexidartinib hydrochloride (such as Turalio), and derivatives thereof.

Exemplary cytokines can include interferons (INFs), interleukins (ILs), and colony stimulating factors. Exemplary INFs can include, but are not limited to, at least one of ropeginterferon alfa-2b-njft (such as Besremi), recombinant Interfon alfa-2b (such as Intron A), interferon-alfa-2a, and peginterferon alfa-2b (such as Sylatron and PEG-intron). Exemplary ILs can include, but are not limited to, aldesleukin (such as Proleukin), interleukin-4, interleukin-2, and derivatives thereof. Exemplary colony stimulating factors can include, but are not limited to, granulocyte-macrophage colony-stimulating factor (such as FM-CSF) and derivatives thereof.

Other exemplary immunologic agents can include, but are not limited to, at least one of tebentafusp-tebn (such as Kimmtrak), plerixafor (such as Mozobil), histamine dihydrochloride (such as Ceplene), mifamurtide (such as Mepact), prednisolone (such as Omnipred, Pred Mild, Pred Forte, Orapred, ODT, Veripred 20, Millipred DP, and Pediapred), polyinosinic: polycytidylic acid, Denileukin diftitox (Ontak), LMB-100, Moxetumomab pasudotox (Lumoxiti), RG7787, DT2219ARL, and derivatives thereof.

Immunologic agents can include combinations of agents. Exemplary combinations can include, but are not limited to, at least one of daratumumab and hyaluronidase-fihj (such as Darzalex faspro), trastuzumab and hyaluronidase-oysk (such as Herceptin hylecta), pertuzumab, trastuzumab, and hyaluronidase-zzxf (such as Phesgo), rituximab and hyaluronidase human (such as Rituxan hycela), tegafur and uracil (such as Uftoral), iodine-131 tositumomab (such as Bexxar), and derivatives thereof.

The immunologic agents herein can be administered at or near the site of the cancerous tumor in a therapeutically effective dose. In other embodiments, immunologic agents can be administered away from the site of the cancerous tumor into the systemic circulation in a therapeutically effective dose. In some embodiments, the immunologic agents can be administered through an intravenous port external to the body. In some embodiments, the immunologic agents can be administered via intravesical infusion into the bladder through a catheter of the patient. In other embodiments, the immunologic agents can be administered orally in the form of a pill or capsule. In other embodiments, the immunologic agents can be topically administered to a patient's skin in the form of a cream or ointment.

### Telomerase Inhibition Agents

One or more telomerase inhibition agents can be suitable for use with the methods and devices described herein. It is contemplated herein, telomerase inhibition agents can be administered simultaneously to a patient receiving electrical therapy, before the patient receives electrical therapy, and/or after the patient receives electrical therapy. It is further contemplated herein, telomerase inhibition agents can be administered to a patient simultaneously to other treatment modalities such as photodynamic therapy, stem cell transplant therapy, cryotherapy, immunotherapy, hormone or biologics therapy, chemotherapy, radiation therapy, gene therapy, and/or surgery. For example, in some embodiments, one or more telomerase inhibition agents can be delivered simultaneously to the administration of an electrical therapy.

In some embodiments, telomerase inhibition drugs can include telomerase inhibitors, alternative lengthening of telomeres (ALT) inhibitors, and telomere-targeting agents. In some embodiments, telomerase inhibitors can include agents that directly target telomerase and prevent it from elongating telomeres. Telomerase inhibitors can include, but is not limited to, GRN163L (such as imetelstat). In some embodiments, ALT inhibitors can include that target the ALT pathway. ALT inhibitors can include, but are not limited to, BIBR1532 and RHPS4. In some embodiments, telomere-targeting agents can include agents that are telomeres which can lead to telomere dysfunction and cell death. In some embodiments, telomere-targeting agents can include, but are not limited to MST-312 and TMPyP4.

The telomerase inhibition agents herein can be administered at or near the site of the cancerous tumor in a therapeutically effective dose. In other embodiments, telomerase inhibition agents can be administered away from the site of the cancerous tumor into the systemic circulation in a therapeutically effective dose. In some embodiments, the telomerase inhibition agents can be administered through an intravenous port external to the body.

### Radiation Therapy

One or more radiation therapies can be suitable for use with the methods and devices described herein. In some embodiments, internal radiation therapies can be used herein. Internal radiation therapies can include brachytherapy, also referred to as radioactive seeding, and systemic therapy. In some embodiments, brachytherapy can include placing radioactive material (such as a radiation wafer) at or near the site of the cancerous tumor. In some embodiments, systemic therapy can include administering orally and/or intravenously, radiopharmaceuticals to a patient. Radiopharmaceuticals can include, but are not limited to, at least one of lutetium Lu 177-Dotatate (such as Lutathera), lutetium Lu 177 vipivotide tetraxetan (such as Pluvicto), lutetium LU 177-edotreotide, sonidegib (such as Odomzo), radium 223 dichloride, and derivatives thereof.

In other embodiments, external beam radiation therapies can be used. External beam radiation therapies include 3D conformal radiation therapy, intensity-modulated radiation therapy, arc-based radiotherapy, image-guided radiotherapy, stereotactic radiosurgery, stereotactic body radiation therapy, intraoperative radiation, and particle therapy. In some embodiments, three-dimensional (3D) conformal radiation therapy can include using advanced imaging techniques, such as a computed tomography (CT) scan or magnetic resonance imaging (MRI) scan to generate 3D images of a cancerous tumor and surrounding tissue and allow for a tailored radiation dose and deliver a radiation beam that conforms with the shape and size of the cancerous tumor. In some embodiments, intensity-modulated radiation therapy can include delivering radiation beams at varying intensities to a cancerous tumor from different angles. In some embodiments, arc-based radiotherapy can include delivering radiation beams of varying intensities to a cancerous tumor, wherein the radiation beams are shaped and modulated in real-time. In some embodiments, image-guided radiotherapy can include using an X-ray, CT scan, and/or MRI scan to generate a 3D image of the cancerous tumor to precisely target radiation beams to the tumor. In some embodiments, stereotactic radiosurgery can include identifying a precise location of a cancerous tumor using a CT scan and/or MRI scan and targeting the cancerous tumor with high-energy radiation beams at a plurality of angles. In some embodiments, stereotactic body radiation therapy can include identifying a precise location of a cancerous tumor using a CT scan and/or MRI scan and delivering radiation to the cancerous tumor at a plurality of angles. In some embodiments, intraoperative radiation can include delivering a high dose of radiation to a cancerous tumor during a surgery in which the cancerous tumor is exposed.

In some embodiments, particle therapy can include delivering high-energy particles to a cancerous tumor to allow the particle energy to be deposited directly into the cancerous tumor. Particle therapy can include proton therapy, carbon ion therapy, and heavy ion therapy. In some embodiments, proton therapy can include generating protons in a particle accelerator and directing the protons into a cancerous tumor. In some embodiments, carbon ion therapy can include directing carbon ions into a cancerous tumor. In some embodiments, heavy ion therapy can include generating heavier ions such as helium, neon, and/or argon to deliver a radiation dose to a cancerous tumor.

In other embodiments, boron neutron capture therapy (BNCT) can be used. BNCT can include administering a boron-containing drug such as boronophenylalanine (BPA) and/or sodium borocaptate (BSH) to a patient and subsequently exposing the patient to neutron beams aimed at or near the cancerous tumor. In some embodiments, the neutron beams can range from 1, 2, 3, 4, 5, 6, 7, 8, 9, 10, 11, or 12 mega-electron volts (MeV), or can be an amount falling in a range within any of the foregoing.

In other embodiments, stereotactic implantation of microspheres can be used. Stereotactic implantation of microspheres can include implanting small radioactive beads or microspheres at or near a site of a cancerous tumor using stereotactic techniques, such as surgical techniques. In some embodiments, biocompatible microspheres, made from glass or resin, can be coated with radioactive substances such a yttrium-90 and/or iodine-131.

In some embodiments, it is believed hyperbaric oxygen therapy (HBOT) can enhance the effectiveness of radiation therapy by increasing oxygenation in the cancerous tumor tissue. HBOT can include placing a patient in a pressurized chamber for a desired length of time and breathing 100% oxygen.

In various embodiments, radiation therapy can include delivering a radiation dose per radiation session of about 1 unit of gray (Gy) to over 80 Gy. In some embodiments, the radiation dose can range from 1 Gy, 2, Gy, 3, Gy, 4 Gy, 5 Gy, 6 Gy, 7 Gy, 8 Gy, 9 Gy, and 10 Gy. In other embodiments, the radiation dose per radiation session can range from 5 Gy, 10 Gy, 15 Gy, 20 Gy, 25 Gy, 30 Gy, 35 Gy, 40 Gy, 45 Gy, 50 Gy, 55 Gy, 60 Gy, 65 Gy, 70 Gy, 75 Gy, and 80 Gy, or can be an amount falling in a range within any of the foregoing. In some embodiments, the radiation dose per session can be about 2 Gy. In various embodiments, a patient can receive a range of 1 to 60 treatment sessions. In some embodiments, the number of treatment sessions can range from 1, 2, 3, 4, or 5 sessions. In other embodiments, the number of treatment sessions can range from 10, 20, 30, 40, 50, or 60 sessions, or can be an amount falling in a range within any of the foregoing.

It is contemplated herein, radiation therapy can be conducted simultaneously to a patient receiving electrical therapy, before the patient receives electrical therapy, and/or after the patient receives electrical therapy. It is further contemplated herein, radiation therapy can be conducted simultaneously to other treatment modalities such as photodynamic therapy, stem cell transplant therapy, cryotherapy, hormone or biologics therapy, chemotherapy, immunotherapy, gene therapy, telomerase inhibition therapy, and/or surgery. In other embodiments, radiation therapy can be conducted before or after the other treatment modalities described herein.

### Cryotherapy

One or more cryotherapies can be suitable for use with the methods and devices described herein. In some embodiments, internal cryotherapies can be used herein. Internal cryotherapies, also known as cryosurgery or cryoablation, can include inserting a specialized probe or catheter at or near the site of the cancerous tumor and cooling the probe using liquid nitrogen, or other cryogenic gas, to freeze and destroy the cancerous cells. In some embodiments, external cryotherapies can be used herein. External cryotherapies can include exposing a portion of a patient's skin to cool temperatures for a desired period of time such that the exposed skin cells are damaged or destroyed. A patient can be exposed to cool temperatures using a spray or swab with a freezing agent such as liquid nitrogen, liquid nitrous oxide, and/or argon gas.

In various embodiments, cryotherapy can include exposing a patient to temperatures that range from −10° C. to −200° C. For example, a patient can be exposed to temperatures that range from −10° C., −20° C., −30° C., −40° C., −50° C., −60° C., −70° C., −80° C., −90° C., −100° C., −110° C., −120° C., −130° C., −140° C., −150° C., −160° C., −170° C., −180° C., −190° C., or −200° C., or can be an amount falling in a range within any of the foregoing.

It is contemplated herein, cryotherapy can be conducted simultaneously to a patient receiving electrical therapy, before the patient receives electrical therapy, and/or after the patient receives electrical therapy. It is further contemplated herein, cryotherapy can be conducted simultaneously to other treatment modalities such as photodynamic therapy, stem cell transplant therapy, immunotherapy, hormone or biologics therapy, chemotherapy, radiation therapy, gene therapy, telomerase inhibition therapy, and/or surgery. In other embodiments, cryotherapy can be conducted before or after the other treatment modalities described herein.

### Stem Cell Transplant Therapy

One or more stem cell transplant therapies can be suitable for use with the methods and devices described herein. Stem cell transplants can include transplanting autologous, allogeneic, and/or syngeneic stem cells. In some embodiments, autologous stem cell transplants include collecting a patient's own stem cells, such as from his/her blood or bone marrow, and returning the stem cells to the patient after a treatment modality such as chemotherapy and/or radiation therapy is conducted. In some embodiments, allogeneic stem cell transplants include collecting stem cells from a genetically matched donor to a patient and infusing the stem cells into the patient's bloodstream after a treatment modality such as chemotherapy and/or radiation therapy is conducted. In some embodiments, syngeneic stem cell transplants include collecting stem cells from a genetically identical twin of a patient and infusing the stem cells into the patient's bloodstream after a treatment modality such as chemotherapy and/or radiation therapy is conducted.

In some embodiments, therapeutic agents described herein can be used to target glioma stem cells. In some embodiments, therapeutic agents can be delivered via injection into the cancerous tumor or systemic delivery that specifically target glioma stem cells.

It is contemplated herein, stem cell transplant therapy can be conducted simultaneously to a patient receiving electrical therapy, before the patient receives electrical therapy, and/or after the patient receives electrical therapy. It is further contemplated herein, stem cell transplant therapy can be conducted simultaneously to other treatment modalities such as photodynamic therapy, immunotherapy, hormone or biologics therapy, and/or surgery. In other embodiments, stem cell transplant therapy can be conducted before or after the other treatment modalities described herein.

### Surgery

One or more surgeries can be suitable for use with the methods and devices described herein. Surgeries can include open surgery and minimally invasive surgery. In some embodiments, open surgery can include making a large incision in a patient's body to access a cancerous tissue site. In some embodiments, minimally invasive surgery can include using specialized tools and cameras to perform surgery on a patient through a small incision or natural orifice of the patient. For example, minimally invasive surgery can include laser interstitial thermal therapy (LITT) and holmium laser enucleation of the prostate (HOLEP). LITT can include inserting a laser probe at or near a site of a cancerous tumor within a patient's body and emitting a high-energy-light to heat and destroy the tissue. The high-energy light can include wavelengths that can be greater than or equal to 600 nm, 700 nm, 800 nm, 900 nm, 1000 nm, 1100 nm, or 1200 nm, or can be an amount falling in a range within any of the foregoing such as from 600 nm to 1200 nm. HOLEP can include inserting a cystoscope into a patient's urethra and up into the prostate gland and using a holmium laser to remove excess tissue from the prostate. The holmium laser can emit light that can include wavelengths that can be greater than or equal to 2000 nm, 2050 nm, 2100 nm, 2150 nm, or 2200 nm, or can be an amount falling in a range within any of the foregoing.

It is contemplated herein, surgery can be conducted simultaneously to a patient receiving electrical therapy, before the patient receives electrical therapy, and/or after the patient receives electrical therapy. It is further contemplated herein, surgery can be conducted simultaneously to other treatment modalities such as photodynamic therapy, immunotherapy, hormone or biologics therapy, radiation therapy, and/or chemotherapy. In other embodiments, surgery can be conducted before or after the other treatment modalities described herein.

### Photodynamic Therapy

One or more photodynamic therapies can be suitable for use with the methods and devices described herein. Photodynamic therapies can include extracorporeal photopheresis and photoimmunotherapy. In some embodiments, extracorporeal photopheresis can include using photosensitizing agents in combination with ultraviolet light to treat a patient. For example, blood can be drawn from a patient and the white blood cells of the patient can be treated with photosensitizing agents before exposing the white blood cells to ultraviolet light and then returning the blood back to the patient. The ultraviolet light can include wavelengths that can be greater than or equal to 300 nm, 310 nm, 320 nm, 330 nm, 340 nm, 350 nm, 360 nm, 370 nm, 380 nm, 390 nm, 400 nm, or 410 nm, or can be an amount falling in a range within any of the foregoing.

In some embodiments, photoimmunotherapy can include injecting a photosensitizing agent into a patient's bloodstream and after a desired period of time, directing a specific wavelength of light at or near the site of the cancerous tumor. Near-infrared light can be used in embodiments herein. The near-infrared light can include wavelengths that can be greater than or equal to 600 nm, 650 nm, 700 nm, 750 nm, 800 nm, 850 nm, 900 nm, 950 nm, or 1000 nm, or can be an amount falling in a range within any of the foregoing.

In various embodiments, the photosensitizing agents can include padelipofin (such as Tookad), porfimer sodium (such as Photofrin), and temoporfin (such as Foscan). It is contemplated herein, photodynamic therapy can be conducted simultaneously to a patient receiving electrical therapy, before the patient receives electrical therapy, and/or after the patient receives electrical therapy. It is further contemplated herein, photodynamic therapy can be conducted simultaneously to other treatment modalities such as surgery, immunotherapy, hormone or biologics therapy, radiation therapy, and/or chemotherapy. In other embodiments, photodynamic therapy can be conducted before or after the other treatment modalities described herein.

### Gene Therapy

One or more additional gene therapies can be suitable for use with the methods and devices herein. In some embodiments, gene therapies can include apoptosis-inducible FADD/MORTI gene transfer, use of deoxycytidine cDNA, use of E-coli gpt gene, use of herpes simplex virus-thymidine kinase, use of drug sensitivity genes, and viral vectors.

In some embodiments, apoptosis-inducible FADD/MORTI gene transfer can include delivering a modified gene that produces the protein FADD/MORTI into a patient to induce apoptosis in cancerous cells. In some embodiments, the modified gene can be delivered using viral vectors such as adenovirus. In other embodiments, the modified gene can be delivered using non-viral vectors such as liposomes, nanoparticles, and polymers, and the like.

In some embodiments, deoxycytidine cDNA can be used alongside cytarabine arabinoside described above, as a sensitizer to enhance the cytotoxic effects of cytarabine arabinoside. In some embodiments, deoxycytidine cDNA can be introduced into cancerous cells that are deoxycytidine kinase deficient, this allows the cells to produce functional deoxycytidine kinase and metabolize deoxycytidine to deoxycytidine triphosphate which can be incorporated into cancerous cells during replication leading to an increased sensitivity to cytarabine arabinoside.

In some embodiments, E-coli gpt gene can be used to deliver the enzyme hypoxanthine-guanine phosphoribosyltransferase (HPRT) into glioma cells. In some embodiments, the HPRT enzyme can convert the prodrug 6-thioxanthine (G-TX) into a toxic metabolite that can kill cancerous cells.

In some embodiments, herpes simplex virus-thymidine kinase is a gene that can be used to encode an enzyme that can convert a non-toxic prodrug, ganciclovir (GCV), into a toxic compound that can kill cancerous cells.

In some embodiments, drug sensitivity genes can be used to make genetically modified cancer cells more susceptible to therapeutic agents. In some embodiments, viral vectors can be used to deliver the genes into cancerous cells. In some embodiments, gene editing techniques such as CRISPR-Cas9 can insert the genes directly into cancerous cells genome. In some embodiments, drug sensitivity genes can include, but are not limited to, thymidine kinase, cytosine deaminase, and dihydrofolate reductase.

In some embodiments, viral vectors containing radiation-inducible promoters can be used alongside radiation therapy to selectively kill tumor cells. In some embodiments, the viral vectors can be delivered via injection at or near a cancerous tumor site, when radiation therapy is administered to a patient, the promoter is activated leading to the expression of a therapeutic gene, such as a toxin or a cytokine leading to the death of the cancerous cells. In some embodiments, the radiation-inducible promoters can include, but are not limited to CC (A/T) 6GG (CArG), early growth response-1 (Egr-1), hypoxia inducible factor-1 (HIF-1), Cip1/Waf1 (p21), and wild-type p53 activated fragment 1 (WAF1).

It is contemplated herein, gene therapy can be conducted simultaneously to a patient receiving electrical therapy, before the patient receives electrical therapy, and/or after the patient receives electrical therapy. It is further contemplated herein, gene therapy can be conducted simultaneously to other treatment modalities such as surgery, immunotherapy, hormone or biologics therapy, radiation therapy, photodynamic, and/or chemotherapy. In other embodiments, gene therapy can be conducted before or after the other treatment modalities described herein.

### Other Therapies

One or more additional therapies can be suitable for use with the methods and devices described herein. In some embodiments, therapies can include, high-intensity focused ultrasound, low-intensity focused ultrasound, nanobots or microrobots, magnetic therapy, modification of bacteria, modification of viruses, encapsulated cells, antisense therapy, and RNA interference (RNAi).

In some embodiments, high-intensity and low-intensity focused ultrasound can include placing an ultrasound transducer on a patient's skin or inserted into his/her body at or near the site of the cancerous tumor and delivering high-frequency or low-frequency sound waves to the tissue. The frequency can be greater than or equal to 0.5 MHz, 1.0 MHz, 1.5 MHz, 2.0 MHz, 2.5 MHz, 3.0 MHz, or 3.5 MHz, or can be an amount falling in a range within any of the foregoing. In some embodiments, lipid-coated microbubble contrast agents can be injected into a patient's bloodstream where the microbubbles circulate and accumulate at the target tissue, because microbubbles are sensitive to ultrasound waves, the treatment effects can be enhanced.

In some embodiments, nanobots or microrobots can include robots typically a few nanometers to a few micrometers in size capable of recognizing and selectively targeting cancerous cells, delivering therapeutic agents, and/or destroying cancerous cells. In some embodiments, nanobots can be used to cross a patient's blood brain barrier (BBB) and deliver one or more therapeutic agents to a patient's brain tissue. For example, the nanobots can be coated with materials (such as transferrin, apolipoprotein, low-density lipoprotein receptors, glucose, and cell-penetrating peptides) that are recognized by receptors on the surface of the BBB cells, allowing the nanobots to be transported across the barrier.

In some embodiments, magnetic therapy can include using magnets positioned external to a patient at or near a cancerous site. Without being bound by theory it is believed magnets can enhance to delivery of cancer drugs to cancerous tumors and stimulate the immune system to attack cancerous cells.

In some embodiments, bacteria can be genetically modified to specifically target and kill cancerous cells. Genetically modified bacteria can include genetically modified bacteria for tumor-specific lysis. In some embodiments, genetically modified bacteria can be injected into a cancerous tumor, inserted into a blood vessel of a tumor using a catheter, and/or administered orally or intravenously. In some embodiments, the bacteria that is genetically modified can include, but is not limited to, Salmonella typhimurium, Escherichia coli, and/or Listeria monocytogenes.

In some embodiments, viruses can be genetically modified to selectively target and kill cancerous cells (oncolytic virotherapy). In some embodiments, genetically modified viruses can be injected into a cancerous tumor or delivered into a patient via intravenous infusion. In some embodiments, a genetically modified virus can infect and replicate within cancerous cells leading to cell death. In some embodiments, the multiplication of the virus can trigger an immune response leading to the further destruction of cancerous cells. In some embodiments, the virus that is genetically modified can include, but is not limited to, neurotrophic viruses such as adenoviruses, herpes simplex virus, and measles virus, vesicular stomatitis virus, poliovirus, Newcastle disease virus, reovirus, adeno-associated virus, vaccina virus, and/or maraba virus.

In some embodiments, encapsulated cells can be engineered to produce therapeutic agents. In some embodiments, cells can be encapsulated in a biocompatible material that protects the cells from a patient's immune system, the cells can then be implanted in a patient's body where the cells can begin to produce and release therapeutic agents described herein.

In some embodiments, antisense therapies can be delivered to cancerous cells via injection into the cancerous tumor or systemically, to stop or slow down the growth of cancerous cells. In some embodiments, antisense therapy can include using short DNA or RNA molecules to bind to and inactivate or degrade messenger RNA molecules that encode proteins involved in cancerous growth and progression.

In some embodiments, RNAi can include delivering RNAi-based therapies directly to a cancerous tumor via injection to silence specific genes by binding messenger RNA and preventing the messenger RNA from being translated into a protein. In some embodiments, small interfering RNAs (SiRNAs), short hairpin RNAs (shRNAs), and/or microRNAs (miRNAs) can be synthesized and delivered into the cancerous tumor using viral vectors, liposomes, and/or nanoparticles.

It is contemplated herein, the therapies listed above can be conducted simultaneously to a patient receiving electrical therapy, before the patient receives electrical therapy, and/or after the patient receives electrical therapy. It is further contemplated herein, the therapies listed above can be conducted simultaneously to other treatment modalities such as surgery, immunotherapy, hormone or biologics therapy, radiation therapy, cryotherapy, gene therapy, telomerase inhibition therapy, and/or chemotherapy. In other embodiments, the therapies listed above can be conducted before or after the other treatment modalities described herein.

### Therapy Sequencing

One or more therapy sequences can be suitable for use with the methods and devices described herein. Therapies can include, but are not limited to, electrical therapy, chemotherapy, cryotherapy, surgery, immunotherapy, photodynamic therapy, stem cell transplant therapy, radiation therapy, gene therapy, telomerase inhibition therapy, hormone/biological therapy, and other therapies described herein.

In various embodiments, one or more therapies can be initiated. In some embodiments, electrical therapy can be administered in combination with chemotherapy. In some embodiments, electrical therapy can be administered in combination with cryotherapy. In some embodiments, electrical therapy can be administered in combination with surgery. In some embodiments, electrical therapy can be administered in combination with immunotherapy. In some embodiments, electrical therapy can be administered in combination with photodynamic therapy. In some embodiments, electrical therapy can be administered in combination with stem cell transplant therapy. In some embodiments, electrical therapy can be administered in combination with radiation therapy. In some embodiments, electrical therapy can be administered in combination with hormone/biological therapy.

In various embodiments, electrical therapy can be administered simultaneously with one or more of the therapies described above. For example, electrical therapy can be administered simultaneously to chemotherapy. In other embodiments, electrical therapy can be administered before one or more of the therapies described above. For example, electrical therapy can be administered before immunotherapy. In other embodiments, electrical therapy can be administered after one or more of the therapies described above. For example, electrical therapy can be administered after surgery. In other embodiments, electrical therapy can be administered between one or more of the therapies described above. For example, electrical therapy can be administered between one or more rounds of chemotherapy. As described herein, a round refers to one of a sequence of sessions of a therapy, where each session is considered a therapeutically effective dose.

In some embodiments, one or more rounds of one or more therapies can be initiated. In various embodiments, multiple rounds of a therapy can be initiated. For example, 1-60 rounds of electrical therapy can be initiated. In some embodiments, 1, 5, 10, 15, 20, 25, 30, 35, 40, 45, 50, 55, or 60 or can be an amount falling in a range within any of the foregoing rounds of the electrical therapy can be initiated. In other embodiments, multiple rounds of each type of therapy can be initiated. For example, 1-60 rounds each of electrical therapy, photodynamic therapy, and chemotherapy can be initiated. In some embodiments, 1, 5, 10, 15, 20, 25, 30, 35, 40, 45, 50, 55, or 60 or can be an amount falling in a range within any of the foregoing rounds of each of the electrical therapy, photodynamic therapy, and chemotherapy can be initiated. In some embodiments, the same number of rounds of each type of therapy can be initiated. In other embodiments, a different number of rounds of each type of therapy can be initiated.

In some embodiments, one or more rounds of each therapy can be initiated for a matter of minutes, hours, days, weeks, or months. For example, one or more rounds of electrical therapy can be initiated for 1 minute, 4 hours, 6 days, 8 weeks, or 10 months, or can be an amount falling in a range within any of the foregoing.

### Applied TTF Electrical Fields

The TTF electrical fields applied to the cancerous tumors using the methods herein can be applied using a variety of modalities. Exemplary therapeutic parameter sets can include those that implement the following concepts: sweeping through a range of frequencies; stacking of one or more frequencies simultaneously; stepping through one or more frequencies sequentially; the spatial or temporal delivery of one or more TTF electrical fields; sweeping through a range of TTF electrical field strengths; applying an effective rotating electric field; modulating a voltage control mode or a current control mode; implementing one or more duty cycles; pulse width modulation; manipulation of the electrical waveform shape and/or pulse sequence; and the occasional use of high frequency or high electric fields strength pulses.

The therapeutic parameter sets can be programmed into a medical device to operate autonomously, or they can be queried and manipulated by the subject or a clinician using an external computation device such as a programmer, a home-based unit, and/or a mobile unit (e.g. a cellular phone, personal computer, smart phone, tablet computer, and the like). In other embodiments, the therapeutic parameter sets can be wirelessly communicated to the medical device from an external computation device. Frequencies and/or TTF electrical field strengths suitable for use in any of the therapeutic parameter sets herein are discussed above with respect to the TTF electrical field generating circuit. In some embodiments, one or more therapeutic parameter sets can be implemented simultaneously. In other embodiments, one or more therapeutic parameter sets can be implemented in an alternating fashion.

By way of example, a TTF electrical field can be applied to the site of a cancerous tumor by sweeping through a range of frequencies. In some embodiments, a frequency sweep can include sweeping from a minimum frequency up to a maximum frequency. In some embodiments, a frequency sweep can include sweeping from a maximum frequency down to a minimum frequency. In other embodiments, sweeping from a minimum frequency up to a maximum frequency and sweeping from the maximum frequency down to the minimum frequency can be repeated as many times as desired throughout the duration of the delivery of the TTF electrical field from the TTF electrical field generating circuit.

As therapy progresses during a frequency sweep, it may be desired to alternate between frequency ranges so that as the cells within a population change in size and number in response to therapy, more cells can be targeted. For example, in some embodiments, a frequency sweep can include alternating between a first frequency sweep covering a range of about 100 kHz to 300 kHz and a second frequency sweep covering a range about 200 kHz to 500 kHz. It will be appreciated that sweeping through a first and second frequency range as described can be performed indefinitely throughout the course of the therapy. In some embodiments, the second frequency sweep (range) can be at higher frequencies than the first frequency sweep (range). In some embodiments, the first frequency sweep (range) can be at higher frequencies than the second frequency sweep (range).

Frequency ranges for the first and second frequency ranges can be any range including specific frequencies recited above with respect to TTF electrical field generating circuit 1609, provided that the lower end of each range is a value less than the upper end of each range. At times, it may be beneficial to have some amount of overlap between the frequency range of the first and second frequency sweep.

### Leads and Electrodes

The leads described herein can be placed into the body at or near the site of a cancerous tumor using a number of techniques. Placement of one or more leads can include using techniques such as transvascular placement, tunneling into the subcutaneous space, and/or surgical placement. In some embodiments, the placement of one or more leads can include placement via one or more natural body orifices. The leads can be placed adjacent to or within a cancerous tumor. In some embodiments, multiple leads can be used near to or far from the cancerous tumor.

In some embodiments one or more leads described herein can be placed in the subcutaneous space. Electrodes on leads placed in the subcutaneous space can be used as the primary near-field generating electrode or as a far-field field generating electrode. In some embodiments, electrodes on leads placed in the subcutaneous space can be used as the primary near-field generating electrode or as a far-field field generating electrode in conjunction with the housing of a medical device. Likewise, one or more leads can be placed transvascularly to act as far-field field generating electrodes in conjunction with an electrode at or near the site of the cancerous tumor or in conjunction with the housing of a medical device.

The leads and electrodes described herein can include additional functional and structural features. In some embodiments, the leads can include those that are compatible with imaging and treatment techniques, including but not limited to MRI (magnetic resonance imaging), X-ray imaging, deep brain stimulation techniques, and/or radiation therapy. In some embodiments, the leads can include one or more conductor cores made from conducting materials. The conductor cores can be formed from conducting materials including metals and/or other conducting materials. Metals can include, but are not limited to, palladium, platinum, silver, gold, copper, aluminum, various alloys including stainless steel, nickel-cobalt alloys such as MP35N® and the like. In some embodiments, the conductor core can be a multifilar coil, including but not limited to a bifilar coil, a trifilar coil, and a quadfilar coil.

In some embodiments, electrodes can be disposed along the length of one or more leads as described herein. Suitable materials for use in the electrodes described herein can include metals such as palladium, to minimize coupling and artifact generation in magnetic fields. In some embodiments, electrodes can be made from other metals and/or other conducting materials. Metals can include, but are not limited to, palladium, platinum, platinum alloys such as platinum-iridium alloy, gold, copper, tantalum, titanium, various alloys including stainless steel, and the like. In some embodiments, electrodes can be in the form of wound coils that can provide an added benefit of increased surface area without compromising flexibility of the electrodes. In some embodiments, the implantable device housing can serve as an electrode.

The leads described herein can also include one or more electrodes disposed along the length of the lead. The leads can include two or more electrodes disposed along the length of the lead. In some embodiments, the electrodes can be tip electrodes found at the distal end of the lead. In other embodiments, the electrodes can be ring electrodes found along the lead but not at the tip of the lead.

In some embodiments, the electrodes can be coil electrodes. In some embodiments, a ring or tip electrode can be positioned in or adjacent to a tumor or cancerous tissue and a coil electrode can be positioned farther from the tumor or cancerous tissue in order to help provide spatial diversity to the generated electric fields. In some embodiments, one or more electrodes can have a length along the lengthwise axis (e.g., proximal to distal axis) of about 0.5, 1, 1.5, 2, 3, 4, 5, 7.5, 10, 15, 20, 30, 40, 50, 75, 100 mm or more. In some embodiments, one or more of the electrodes can have a length falling within a range wherein any of the foregoing distances can serve as the upper or lower bound of the range, provided that the upper bound is greater than the lower bound.

The leads can be unipolar, bipolar, or multipolar. In some embodiments, a unipolar lead can include a lead that generates an TTF electrical field between one electrode and the housing of the medical device. In some embodiments, a bipolar lead can include a lead that can generate a TTF electrical field between two electrodes disposed along the lead, or between both electrodes and the housing of the medical device. In some embodiments, a multipolar lead can include a lead that can generate a TTF electrical field between the more than two electrodes disposed along the lead, between more than two electrodes and the housing of the medical device, or any number of combinations of configurations of electrodes and the housing of the medical device.

The leads herein can include one or more optical emitters along the length of the lead. Optical emitters suitable for use herein can include those that emit light that falls anywhere along the visible spectrum from about 350 nm to 950 nm. Suitable optical emitters can include light emitting diodes or laser diodes. Suitable LEDs can be made from one or more of gallium arsenide (GaAs), gallium phosphide (GaP), gallium arsenide phosphide (GaAsP), silicon carbide (SiC) or fallium indium nitride (GalnN). In some embodiments, the LEDs suitable for use herein can include an LED capable of emitting only one color, or a mono-color LED; an LED capable of emitting two colors, or a bi-color LED; an LED capable of emitting three colors, or a tri-color LED; or an LED capable of emitting more than three colors. The LEDs can be in electrical communication with control circuitry within the housing of the medical devices described herein. In some embodiments, one or more laser diodes can be included along the leads herein, and the laser diodes can be in optical communication with one or more optical fibers disposed within the leads and used for transmitting light from a laser source to a laser diode.

The electrodes suitable for use here can be made of conductive polymers such as carbon filled silicone, polyacetylene, polypyrrole, polyaniline, polytiophene, polyfuran, polyisoprene, polybutadiene, polyparaphenylene, and the like. In other embodiments, the electrodes can be insulated. In some embodiments, the insulation surrounding and electrode can include microporous insulators to prevent cellular apposition, yet still allow for current flow. Microporous insulators can be made from a number of the insulating materials described herein, including but not limited to polytetrafluoroethylene (ePTFE), polyethylene-co-tetrafluoroethene (ETFE), polyurethanes, silicones, poly(p-xylylenc) polymers such as Parylene polymers, polyether block amides such as PEBAX®, nylons, or derivatives thereof. In some embodiments, the electrodes can be coated with various materials, including but not limited to hydrogels or fractal coatings such as iridium oxide, titanium oxide, tantalum pentoxide, other metal oxides, poly(p-xylylene) polymers such as Parylene, and the like.

A number of lead fixation techniques and configurations can be used in accordance with the embodiments herein. Some non-limiting examples of lead fixation techniques can include biocompatible glue fixation, talon fixation, helix coil fixation, passive centering of the lead in the vascular system, tine fixation within the localized vascular system, spiral bias fixation within the localized vascular system, compression fixation, suture sleeve fixation, and the like. In some examples, the leads embodied herein can be placed within the vascular system surrounding or adjacent to the site of the cancerous tumor. In other embodiments, the leads embodied herein can be place surgically at or within or surrounding the site of the cancerous tumor.

The leads suitable for use herein can also include one or more open lumens that run the entire longitudinal length of, or a select portion of the longitudinal length of the lead. In some embodiments, the open lumen can include an integrated biopsy apparatus suitable for obtaining biopsy samples from a cancerous tumor site on a periodic basis to monitor disease progression and/or regression. Leads having an open lumen can also be configured to include an integrated drug delivery lumen that can deliver one or more drugs, such as steroids or chemotherapy agents, to the site of the tumor in a single bolus or periodically via a metered pump. The leads can include one or more portals disposed along the length of the lead to provide an outlet for drug delivery at or near the site of a cancerous tumor.

In some embodiments a portion of the lead or the entire lead can include a drug eluting coating. In some embodiments, the drug eluting coating can include an anti-inflammatory agent, such as a steroid. In some embodiments, the steroid can be dexamethasone. In other embodiments, the drug eluting coating can include a chemotherapy agent. In some embodiments, the chemotherapy agent can include a taxane or derivatives thereof, including but not limited to paclitaxel, docetaxel, and the like. In other embodiments, the drug eluting coating can be configured to release additional classes of chemotherapy agents, including, but not limited to alkylating agents, plant alkaloids such as vinca alkaloids, cytotoxic antibiotics, topoisomerase inhibitors, and the like. In some embodiments, the drug eluting coating can be configured to release the drug from the coating in a time-release fashion.

The leads herein can adopt a number of shapes or configurations. In some embodiments, the leads can be linear and in other embodiments the leads can be circular. A circular lead may be a completely closed loop or it may be a semi-closed loop. In some embodiments, the lead can include a bendable core that can allow the lead to be shaped into many configurations, including but not limited to a U shape, an S shape, a spiral shape, a half circle, an oval, and the like.

In yet other examples, the leads suitable for use herein can include fluorimetric or magnetic markers that can assist the clinician in precise placement at or near the site of a cancerous tumor. The leads can also include integrated pH sensors for detecting the change in the pH at or near the cancerous tumor or other chemical sensors suitable for analyzing the concentration of a chemical analyte of interest.

### TTF Electrical Field Generators

The medical devices embodied herein can include TTF electrical field generators particularly suited for therapeutic and diagnostic techniques used during the course of treatment for a cancerous tumor. In some embodiments, the TTF electrical field generators suitable for use herein can include those that have been treated by radiation hardening to make the components resistant to the damaging effects of radiation therapy treatments often prescribed as a main line treatment for cancerous tumors. TTF electric field generators can include components such as those described in reference to FIGS. 3 and 5 above.

TTF electrical field generators embodied herein can be programmed with any number of therapeutic parameter sets as described. The TTF electrical field generators can be programmed prior to implant, or they can be programmed by a clinician using an external computation device such as a programmer, a home-based unit, and/or a mobile unit (e.g. a cellular phone, personal computer, smart phone, tablet computer, and the like). In some embodiments, therapy parameters can be delivered to the TTF electrical field generator via a telemetry circuit. In some embodiments, the TTF electrical field generator can include a recharge circuit communicatively coupled to a receiver coil to facilitate transcutaneous recharging of the medical device. In some embodiments, the TTF electrical field generator can communicate wirelessly between the receiver coil and an external charging device.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. A method for providing cancer therapy comprising:
applying one or more TTF electrical fields at or near a site of a cancerous tumor, a surgical excision site, and/or a targeted therapy site of a subject; and
administering a non-electrical field based cancer therapy to the subject; and
wherein applying the TTF electrical fields is performed before, after, and/or simultaneously with administering the non-electrical field based cancer therapy.

2. The method for providing cancer therapy of claim 1, wherein applying the TTF electrical fields is performed both before and after administering the non-electrical field based cancer therapy.

3. The method for providing cancer therapy of claim 1, wherein applying the TTF electrical fields is performed simultaneously with administering the non-electrical field based cancer therapy.

4. The method for providing cancer therapy of claim 1, wherein the TTF electrical fields are applied at a first intensity simultaneously with administering the non-electrical field based cancer therapy and then at a second intensity after administering the non-electrical field based cancer therapy ceases.

5. The method for providing cancer therapy of claim 1, wherein the TTF electrical fields are applied for a first time period simultaneously with administering the non-electrical field based cancer therapy and then for a second time period after administering the non-electrical field based cancer therapy ceases, wherein the second time period is longer than the first time period.

6. The method for providing cancer therapy of claim 1, wherein administering a non-electrical field based cancer therapy to the subject comprises administering a chemotherapeutic agent, wherein the chemotherapeutic agent is at least one of an antimitotic agent, an alkylating agent, an antimetabolite, a kinase inhibitor, a proteasome inhibitor, a histone deacetylase inhibitor, a topoisomerase inhibitor, a poly(ADP-ribose) polymerase (PARP) inhibitor, an antitumor antibiotic, or a retinoid.

7. The method for providing cancer therapy of claim 1, wherein administering a non-electrical field based cancer therapy to the subject comprises administering an immunotherapeutic agent to the subject, wherein the immunotherapeutic agent is at least one of a monoclonal antibody, an adoptive cell therapeutic agent, a cancer vaccine, an immunomodulatory agent, or a cytokine.

8. The method for providing cancer therapy of claim 1, wherein administering a non-electrical field based cancer therapy to the subject comprises administering a hormone/biologic therapeutic agent to the subject, wherein the hormone/biologic therapeutic agent is at least one of a progestin, a steroidal antiestrogen, a luteinizing hormone-releasing hormone agonist, a gonadotropin-releasing hormone agonist, an antiandrogen, an aromatase inhibitor, a selective estrogen receptor modulator, a corticosteroid, a somatostatin analogue, a prolactin lowering agent, or a thyrotropin stimulating hormone agonist.

9. The method for providing cancer therapy of claim 1, wherein administering a non-electrical field based cancer therapy to the subject comprises administering a telomerase inhibition agent to the subject, wherein the telomerase inhibition agent is a telomerase inhibitor, an alternative lengthening of telomeres (ALT) inhibitor, or a telomere-targeting agent.

10. The method for providing cancer therapy of claim 1, wherein administering a non-electrical field based cancer therapy to the subject comprises administering radiation to the subject.

11. The method for providing cancer therapy of claim 10, wherein the radiation is administered at a radiation dose selected from a range of between 1 Gy to 80 Gy.

12. The method for providing cancer therapy of claim 1, wherein administering a non-electrical field based cancer therapy to the subject comprises applying subzero temperatures to the subject, wherein the subzero temperatures are from −10° C. to −200° C.

13. The method for providing cancer therapy of claim 1, wherein administering a non-electrical field based cancer therapy to the subject comprises transplanting one or more stem cells into the subject.

14. The method for providing cancer therapy of claim 1, wherein administering a non-electrical field based cancer therapy to the subject comprises performing surgery on the subject.

15. The method for providing cancer therapy of claim 1, wherein administering a non-electrical field based cancer therapy to the subject comprises performing laser therapy, wherein the laser therapy includes applying one or more laser beams to the subject at a wavelength of 600 nm to 1200 nm.

16. The method for providing cancer therapy of claim 1, wherein administering a non-electrical field based cancer therapy to the subject comprises administering at least one selected from the group consisting of chemotherapy, radiation therapy, cryotherapy, surgery, immunotherapy, photodynamic therapy, stem cell transplant therapy, hormone/biological therapy, telomerase inhibition therapy, and gene therapy.

17. The method for providing cancer therapy of claim 1, wherein the one or more TTF electrical fields are applied at frequencies selected from a range of 100 kHz to 300 kHz.

18. The method for providing cancer therapy of claim 1, wherein the one or more TTF electrical fields are applied at field strengths of 1 V/cm to 10 V/cm.

19. The method for providing cancer therapy of claim 1, wherein the one or more TTF electrical fields are applied using electrodes that are external to the patient.

20. The method for providing cancer therapy of claim 1, wherein the one or more TTF electrical fields are applied using electrodes that are implanted within the patient.
